(19) United States
(12) Patent Application Publication
(10) Pub. No.: US 2003/0144219 A1
(43) Pub. Date: Jul. 31, 2003

(54) Formulations and methods for treatment or amelioration of inflammatory conditions

(76) Inventors: Stephen Dodge Phinney, Elk Grove, CA (US); Darlene M. Dreon, Menlo Park, CA (US)

Correspondence Address: GALILEO PHARMACEUTICALS, INC., (PREVIOUSLY GALILEO LABORATORIES, INC.), 5301 PATRICK HENRY DRIVE, SANTA CLARA, CA, 95954, US

(21) Appl. No.: 10/295,493

(22) Filed: Nov. 15, 2002

Related U.S. Application Data
(60) Provisional application No. 60/335,545, filed on Nov. 15, 2001.

(51) Int. Cl. A61K 31/7048; A61K 31/353; A61K 31/355; A61K 31/202

(52) U.S. Cl. 514/27; 514/456; 514/458; 514/560

(57) ABSTRACT

Formulations and methods for the treatment and/or amelioration of symptoms of inflammatory conditions and associated systemic inflammatory responses are described herein. The compositions comprise a non-alpha tocopherol (especially gamma-, beta-, or delta-tocopherol) and one or more of an omega-3 fatty acid, such as docosahexaenoic acid (DHA) or a flavonoid.

### CROSS-REFERENCE TO RELATED APPLICATIONS

This application claims benefit of U.S. Provisional Patent Application Serial No. 60/335,545 filed Nov. 15, 2001, which is incorporated by reference herein in its entirety.

### TECHNICAL FIELD

The present invention relates to formulations or compositions comprising a non-alpha tocopherol, such as gamma-tocopherol, beta-tocopherol, and/or delta-tocopherol, in a formulation that includes at least one other component, such as a flavonoid or a highly unsaturated fatty acid, such as for example, all-cis 4,7,10,13,16,19-docosahexaenoic acid (DHA). In some embodiments, the formulation may also include a mineral, such as magnesium. In some embodiments, the formulation further comprises nutritional excipients and, in other embodiments, pharmaceutical excipients. The present invention also relates to methods for the treatment and/or amelioration of various inflammatory conditions and their associated systemic inflammatory response.

### BACKGROUND

Inflammation and associated inflammatory responses are important components of host protection to a variety of insults, which may be infectious or non-infectious in nature. While specific responses to an injury or insult may vary, the “inflammatory response” can be viewed as a composite response including successive events in response to a stimulus. Thus, inflammation involves a number of cellular, molecular and physiologic events. These events include vasodilatation; increased vascular permeability; extravasation of plasma leading to interstitial edema; chemotaxis of neutrophils, macrophages and lymphocytes; cytokine production; increased acute phase reactants; leukocytosis; fever; increased metabolic rate; impaired albumin production and hypoalbuminemia; activation of complement; and stimulation of antibodies.

Inflammation is associated with many different diseases or disorders such as, for example, neurodegenerative diseases, diabetes-associated nephropathy and retinopathy, protein wasting, muscle fatigue or inflammation, infectious diseases, as well as various cardiovascular diseases or disorders, including atherosclerosis; neurodegenerative diseases such as, Alzheimer's disease; infectious disease, such as, for example, myocarditis, cardiomyopathy, acute endocarditis, pericarditis; Systemic Inflammatory Response Syndrome (SIRS)/sepsis; adult respiratory distress syndrome (ARDS); asthma; rheumatoid arthritis, osteoarthritis, systemic lupus erythematosis; airway hyperresponsiveness (AHR); bronchial hyperreactivity; chronic obstructive pulmonary disease (COPD); congestive heart failure (CHF); inflammatory complications of diabetes mellitus; metabolic syndrome, end stage renal disease (ESRD); as well as a variety of dermal conditions.

A number of proximal mediators of the inflammatory response have been identified. These include the inflammatory cytokines, interleukin-1 through 17, including interleukin-1α (IL-1α), interleukin-1β (IL-1β), and tumor necrosis factor alpha (TNF-α). Other molecules have been reported for use as markers of systemic inflammation, including for example, CRP; certain cellular adhesion molecules such as e-selectin (also known as ELAM), sICAM-1 (U.S. Pat. No. 6,049,147), integrins, ICAM-1, ICAM-3, BL-CAM, LFA-2, VCAM-1, NCAM, PECAM, and neopterin; and B61 (U.S. Pat. No. 5,688,656). Other markers associated with inflammation include leukotriene, thromboxane, and isoprostane. Other proteins or markers associated with inflammation include serum amyloid A protein, fibrinectin, fibrinogen, leptin, prostaglandin E2, serum procalcitonin, soluble TNF receptor 2 (sTNFr2), and elevated white blood count, including percent and total granulocytes (polymorphonuclear leukocytes) monocytes, lymphocytes and eosinophils, and increased erythrocyte sedimentation rate. Further indicators of inflammatory states, particularly in ESRD patients, may include decreased levels of pre-albumin and albumin.

C-reactive protein (CRP) has recently gained recognition as a marker for inflammatory conditions, including risk of cardiovascular disease. See U.S. Pat. No. 6,040,147. In humans CRP levels are elevated in response to infection, trauma, surgery, and tissue infarction. The magnitude of the increase varies from about 50% to as much as 100-fold during systemic inflammation (Gabay, C., et al., New Engl. J. Med. 340:448-454, 1999). Most CRP production is from hepatocytes in response to pro-inflammatory cytokines, especially interleukin-6 and 1β (Ganter, U., et al., EMBO J. 8: 3773-3779, 1989), although macrophages have also been reported to release CRP (Dong, Q, et al, J. Immunol. 156: 481504820,1996).

There remains a need for effective compositions and methods for treating and/or ameliorating the symptoms of inflammation. Further, in view of risk factors associated with CRP, there is a need for methods for reducing elevated CRP levels associated with inflammation associated with inflammatory conditions, such as those described herein.

The disclosure of all patents and publications cited herein are incorporated by reference in their entirety.

### DISCLOSURE OF THE INVENTION

The present invention relates to compositions and methods for the treatment and/or amelioration of inflammatory conditions and their associated systemic inflammatory response(s) in a mammalian subject. Inflammatory conditions that can be addressed by formulations and methods of the present invention include, but are not limited to neurodegenerative diseases, diabetes-associated nephropathy and retinopathy, protein wasting, muscle fatigue or inflammation, infectious diseases, as well as various cardiovascular diseases or disorders, including atherosclerosis; neurodegenerative diseases such as, Alzheimer's disease; infectious disease, such as, for example, myocarditis, cardiomyopathy, acute endocarditis, pericarditis; Systemic Inflammatory Response Syndrome (SIRS)/sepsis; adult respiratory distress syndrome (ARDS); asthma; rheumatoid arthritis, osteoarthritis, systemic lupus erythematosis; airway hyperresponsiveness (AHR); bronchial hyperreactivity; chronic obstructive pulmonary disease (COPD); congestive heart failure (CHF); inflammatory complications of diabetes mellitus; metabolic syndrome, end stage renal disease (ESRD); as well as a variety of dermal conditions.

In one embodiment, the present invention provides anti-inflammatory formulations comprising a non-alpha tocopherol (including, without limitation, beta-tocopherol, gamma-tocopherol and/or delta-tocopherol or metabolites thereof, singly or in combination) and either an omega-3 fatty acid, a flavonoid or a combination of an omega-3 fatty acid and a flavonoid. In some embodiments, the formulation will also include a mineral, such as magnesium (Mg2+).

The present invention provides non-alpha-tocopherol-enriched formulations, as outlined above, and methods for using such formulations in the treatment and/or amelioration of a symptom of inflammation or a symptom of an inflammatory condition and/or for reducing the level of an inflammatory marker associated with inflammation or an inflammatory condition and/or for reducing a symptom associated with inflammation or an inflammatory condition, such as pain and edema. In some examples, the present invention provides compositions and methods for reducing one or more biochemical markers of inflammation, including for example reducing CRP or reducing IL-6 or reducing white blood cell count, thereby ameliorating an inflammatory symptom associated with disease or an inflammatory condition and/or reducing a mammalian subject's risk of progressing into long term or chronic inflammatory conditions. In some examples, the present invention provides compositions and methods for maintaining normal or healthy levels of inflammatory markers in subjects.

In another embodiment, the invention is directed to a method of reducing the level of an inflammatory biomarker in an individual subject to an inflammatory condition. According to this feature of the invention, a formulation comprising a non-alpha tocopherol and an omega-3 fatty acid is administered to the individual. More generally, the inflammatory biomarker can be any suitable biomarker known or recognized as being related to the inflammatory condition, including but not limited to: inflammatory cytokines, interleukin-1 through 17, including interleukin-1α (IL-1α), interleukin-1β (IL-1β), tumor necrosis factor alpha (TNF-α); markers of systemic inflammation, including for example, CRP; certain cellular adhesion molecules such as e-selectin, integrins, ICAM-1, ICAM-3, BL-CAM, LFA-2, VCAM-1, NCAM, PECAM, and neopterin; and B61; leukotriene, thromboxane, isoprostane, serum amyloid A protein, fibrinectin, fibrinogen, leptin, prostaglandin E2, serum procalcitonin, soluble TNF receptor 2 (sTNFr2), erythrocyte sedimentation rate, erythema; elevated white blood count (WBC), including percent and total granulocytes (polymorphonuclear leukocytes) monocytes, lymphocytes and eosinophils; and increased erythrocyte sedimentation rate. Further biomarkers of inflammatory states may include decreased levels of pre-albumin and albumin.

In one embodiment, the biomarker will be one or more of C-reactive protein (CRP), interleukin-1-alpha (IL-1-alpha), interleukin-1-beta (IL-1-beta), interleukin-6 (IL-6), and elevated white blood cell count (WBC).

The omega-3 fatty acid in the formulation may comprise docosahexaenoic acid (DHA); preferably, according to a further embodiment, the DHA: eicosapentaenoic (EPA) ratio of such a formulation will be greater than 10:1 (DHA:EPA). According to a further embodiment, the formulation will be essentially free of EPA.

Non-alpha tocopherols for use in the formulations of the invention may be any of a number of tocopherols (including mixed tocopherols), with a preference that the amount of alpha tocopherol present in the formulation will be less than 25%, and preferably less than 10% alpha tocopherol (where percentage is measured against all tocopherols present in the formulation). According to a further feature, the non-alpha-tocopherol is selected from the group consisting of gamma-tocopherol, a gamma-tocopherol metabolite such as gamma-carboxy ethyl chroman (gamma-CEHC), beta-tocopherol, a beta-tocopherol metabolite, delta-tocopherol and delta-tocopherol metabolite, or may be a mixture of any of the foregoing tocopherols.

According to still a further embodiment, the formulation may also include a flavonoid. Alternatively, the formulation may comprise a non-alpha tocopherol and a flavonoid. In either case, although any of a number of flavonoids will be found to provide the desired characteristics, particularly preferred flavonoids are quercetin, hesperetin, or a mixture of quercetin and hesperetin. Other mixtures or formulations can be readily selected in accordance with the teachings set forth in the specification.

In a further embodiment, formulations of the invention may include a mineral component. An exemplary mineral component is magnesium, although any of a number of components may be selected, in accordance with the present invention.

While the formulations of the invention are useful in countering the symptoms and effects of most inflammatory conditions, it is understood that particularly preferred conditions are those that are characterized by an elevation of one or more of the following biomarkers: C-reactive protein (CRP), interleukin-6 (IL-6), interleukin-1-alpha (IL-1-alpha), interleukin-1-beta (IL-1-beta), erythrocyte sedimentation rate, and white blood cell count (WBC).

Further biomarkers are described in the specification and known in the art.

According to a preferred embodiment, certain inflammatory conditions and symptoms thereof, particularly associated biomarkers, are particularly amenable to amelioration, treatment or alteration, as the case may be, by formulations of the present invention. These include muscle inflammation and associated biomarkers CRP, IL-6, erythrocyte sedimentation rate, and elevated white blood cell count (WBC); end-stage renal disease (ESRD) and associated biomarkers CRP, IL-6, IL-1 (alpha and beta), soluble TNF receptor 2 (sTNFr2), as well as lowered levels of pre-albumin and albumin; and diabetes, particularly type II diabetes, and associated biomarkers hemoglobin A1c (HbA1c), CRP, and IL-6; cardiovascular disease, particularly associated with the biomarker CRP; and metabolic syndrome, particularly associated with elevated triglycerides and CRP. Other inflammatory indications and associated biomarkers will be apparent to persons skilled in the art.

In a related embodiment, the invention is directed to a method for ameliorating a symptom of an inflammatory condition in an individual subject to an inflammatory condition. According to this aspect of the invention, a formulation comprising a non-alpha-tocopherol and an omega-3 fatty acid is administered to the subject. Symptoms that are addressed according to this aspect of the invention include elevated biomarkers, such as, for example, C-reactive protein (CRP), interleukin-6 (IL-6), interleukin-1-alpha (IL-1-alpha), interleukin-1-beta (IL-1-beta), erythrocyte sedimentation rate, and white blood cell count (WBC); edema, diminished biomarkers such as, for example, pre-albumin or albumin, pain, and other symptoms of inflammation.

Formulations useful in this aspect of the invention are similar to the non-alpha-tocopherol/omega-3 fatty acid formulations described above. Non-alpha tocopherols are preferably selected from the group consisting of gamma-tocopherol, a gamma-tocopherol metabolite, such as gamma-CEHC, beta-tocopherol, a beta-tocopherol metabolite, delta-tocopherol and delta-tocopherol metabolite. Particularly favored omega-3 fatty acids include DHA, particularly DHA that is essentially free of EPA. Other components may include a flavonoid, such as quercetin, hesperetin, or a mixture of quercetin and hesperetin and/or magnesium. According to a related aspect of the invention, a non-alpha tocopherol may be mixed with a flavonoid to provide beneficial anti-inflammatory effects, as well. Such formulations are useful in treating or ameliorating the symptoms of a wide variety of inflammatory conditions, including, but not limited to the inflammatory conditions listed above, and particularly including muscle inflammation, ESRD, diabetes, cardiovascular disease and metabolic syndrome.

According to another related embodiment, the invention includes anti-inflammatory formulations having the components listed above. More particularly, the invention includes the use of a formulation consisting of a non-alpha tocopherol and an omega-3 fatty acid in the manufacture of a medicament for the reduction of a symptom of an inflammatory condition. Symptoms of inflammatory conditions include the symptoms listed above, such as pain and edema, and particularly biomarkers selected from the group consisting of C-reactive protein (CRP), interleukin-1-alpha (IL-1-alpha), interleukin-1-beta (IL-1-beta), interleukin-6 (IL-6), and white blood cell count (WBC).

In accordance with this aspect of the invention, the medicament is made to include a non-alpha-tocopherol selected from the group consisting of gamma-tocopherol, a gamma-tocopherol metabolite, particularly gamma-CEHC, beta-tocopherol, a beta-tocopherol metabolite, delta-tocopherol and delta-tocopherol metabolite. A particularly useful omega-3 fatty acid for use in the medicament is docosahexaenoic acid (DHA), particularly where the formulation contains very little EPA relative to the DHA (less than 1:10, EPA:DHA). Medicaments may further contain a flavonoid, selected as described above, particularly quercetin, hesperetin, or a mixture of quercetin and hesperetin. Alternatively, in some cases, a useful medicament may comprise a non-alpha tocopherol in conjunction with a flavonoid, in the absence of DHA. Other components may be added, for example a mineral, such as magnesium, and/or alpha lipoic acid. Inflammatory conditions that are amenable to treatment with such a medicament include the ones listed above, and, in particular, ESRD, diabetes, cardiovascular disease, metabolic syndrome and muscle inflammation or fatigue.

It is appreciated that the components of the formulations of the invention may be administered as a single administration or packaged unit or in two or more administrations or packaged units.

These and other objects and features of the invention will become more fully apparent when the following detailed description considered.

### BRIEF DESCRIPTION OF THE DRAWINGS

NOT APPLICABLE

### MODE FOR CARRYING OUT THE INVENTION

I. Definitions

“Inflammation” or “inflammatory symptoms” refers to one or more biological and physiological sequelae including: vasodilatation; increased vascular permeability; extravasation of plasma leading to interstitial edema; chemotaxis of neutrophils, macrophages and lymphocytes; cytokine production; acute phase reactants; C-reactive protein (CRP); increased erythrocyte sedimentation rate; leukocytosis; fever; increased metabolic rate; impaired albumin production and hypoalbuminemia; activation of complement; and stimulation of antibodies.

Inflammation is associated with a number of diseases, disorders and conditions such as for example, cardiovascular diseases or disorders; neurodegenerative diseases such as, Alzheimers; infectious diseases, such as, for example, myocarditis, cardiomyopathy, acute endocarditis, pericarditis; atherosclerosis; Systemic Inflammatory Response Syndrome (SIRS)/sepsis; adult respiratory distress syndrome (ARDS); asthma; rheumatoid arthritis, osteoarthritis, systemic erythematosis (SLE); Airway hyperresponsiveness (AHR); bronchial hyperreactivity; Chronic Obstructive Pulmonary disease (COPD); Congestive Heart Failure (CHF); inflammatory complications of diabetes mellitus; metabolic syndrome; end-stage renal disease (ESRD); muscle fatigue or inflammation and dermal conditions. As used herein, the foregoing listed conditions, and any conditions that have as a symptom inflammation, are encompassed by the term “systemic inflammatory condition” or “inflammatory condition.” As used herein, “respiratory inflammatory conditions” refer to inflammatory conditions that primarily affect the lungs, for example, SIRS, ARDS, asthma and AHR.

Elevated levels of C-reactive protein (CRP) have been associated with various inflammatory conditions. As used herein, “CRP-associated inflammation” refers to inflammatory conditions and/or inflammation associated with elevated levels of CRP such as for example, cardiovascular diseases or disorders, including atrial fibrillation, unstable angina, coronary artery disease, peripheral artery disease, cardiac allograft vasculopathy (CAVD); mastitis; pre-eclampsia; inflammatory bowel conditions; stroke; tissue infarction; lumbosciatica; estrogen/progestin hormone replacement therapy (HRT); infection (bacterial, viral and protozoan); bacterial meningitis; trauma; surgery; biomaterial implants; smoking; obesity; neurodegenerative diseases such as, Alzheimers; infectious disease, such as, for example, myocarditis, cardiomyopathy, acute endocarditis, pericarditis; atherosclerosis; SIRS/sepsis; adult respiratory distress syndrome ARDS; asthma; rheumatoid arthritis, osteoarthritis, systemic lupus erythematosis (SLE); AHR; bronchial hyper-reactivity; COPD; CHF; inflammatory complications of diabetes mellitus type I and type II; metabolic syndrome; end stage renal disease (ESRD), muscle fatigue or inflammation; multiple organ dysfunction syndrome (MODS); aging; acute allergic reactions; gingivitis and dermal conditions.

As used herein, “cardiovascular disease” includes diseases associated with the cardio-pulmonary and circulatory systems including but not limited to ischemia, angina, edematous conditions, artherosclerosis, CHF, LDL oxidation, adhesion of monocytes to endothelial cells, foam-cell formation, fatty-streak development, platelet adherence, and aggregation, smooth muscle cell proliferation, reperfusion injury, high blood pressure, and thrombolic disease.

As used herein, a “symptom” of an inflammatory condition includes physical symptoms (pain, edema, erythema, and the like) associated with a particular inflammatory condition, and/or biomarkers associated either generally with inflammation or particularly with a specific inflammatory condition.

As used herein, “markers associated with inflammation” or “inflammatory biomarkers” include, but are not limited to CRP, cytokines associated with inflammation, such as members of the interleukin family, including IL-1 through IL-17 that are associated with inflammation, TNF-alpha; B61; certain cellular adhesion molecules, such as for example, e-selectin (also known as ELAM), sICAM, integrins, ICAM-1, ICAM-3, BL-CAM, LFA-2, VCAM-1, NCAM and PECAM; neopterin; serum procalcitonin; leukotriene, thromboxane, and isoprostane. In particular, elevated levels of CRP are associated with cardiovascular diseases and disorders, infectious diseases, such as, myocarditis, cardiomyopathy, acute endocarditis, or pericarditis; SIRS; diabetes; metabolic syndrome; muscle fatigue, injury or inflammation; and systemic inflammation. By way of example but not limitation: Elevated levels of IL-6, sTNFr2 and CRP are associated with type II diabetes, muscle inflammation and ESRD; elevated levels of cellular adhesion molecules are associated with systemic inflammation; elevated levels of IL-1 and TNF-alpha are associated with IDDM and NDDM associated inflammation; elevated levels of IL-10 and IL-6 are associated with SIRS; elevated levels of neopterin are associated with SIRS; elevated levels of procalcitonin are associated with systemic inflammation. Other proteins or markers associated with inflammation include serum amyloid A protein, fibrinectin, fibrinogen, leptin, prostaglandin E2, serum procalcitonin, soluble TNF receptor 2, elevated erythrocyte sedimentation rate, and elevated white blood count, including percent and total granulocytes (polymorphonuclear leukocytes)m monocytes, lymphocytes and eosinophils.

A “formulation” refers to a combination of active components or ingredients that are administered together or separately under a coordinated dosing regimen. For purposes of the present invention, a formulation need not consist of admixed components. Rather, it may include components that are given separately in different oral forms or even via different modes of administration, for example as a combination of oral and parenteral treatments. A formulation may also comprise a “kit” whereby components are bundled together in a combination packaging format.

By “tocopherol” is meant any of a family of molecules which are characterized by a 6-chromanol ring structure and a side chain at the 2 position. A “non-alpha-tocopherol enriched tocopherol composition”, as used herein refers to the non-alpha-tocopherol, such as for example, gamma-, beta- or delta-tocopherol as being enriched with respect to total tocopherols in the composition. Tocopherols possess a 4′,8′,12′-trimethyltridecyl phytol side chain. As used herein, the term “tocopherol” encompasses, but is not limited to: alpha-tocopherol, beta-tocopherol, gamma-tocopherol, delta-tocopherol, epsilon-tocopherol, [R-(E,E)]-3,4-dihydro-2,5,8-trimethyl-2-(4,8,12-trimethyl-3,7,11-tridecatrienyl)-2H-1-benzopyran-6-ol; 2,5,8-trimethyl-2-(4,8,12-trimethyltrideca-3,7,11-trienyl)chroman-6-ol; 5-methyltocol; zeta₁-tocopherol, 3,4-dihydro-2,5,7,8-tetramethyl-2-(4,8,12-trimethyl-3,7,11-tridecatrienyl)-2H-1-benzopyran-6-ol; 2,5,7,8-tetramethyl-2-(4,8,12-trimethyl-3,7,11-tridecatrienyl)-6-chromanol; 5,7,8-trimethyltocotrien-3′,7′,11′-ol; zeta₂-tocopherol, 3,4-dihydro-2,5,7-trimethyl-2-(4,8,12-trimethyltridecyl)-2H-1-benzopyran-6-ol; 2,5,7-trimethyl-2-(4,8,12-trimethyltridecyl-6-chromanol; 5,7-dimethyltocol; and eta-tocopherol, 3,4-dihydro-2,7-dimethyl-2-(4,8,12-trimethyltridecyl)-2H-1-benzopyran-6-ol; 2,7-dimethyl-2-(4,8,12-trimethyltridecyl)-6-chromanol; 7-methyltocol. Other tocopherols include xi₁-, xi₂-, and sigma-tocopherols.

Generally speaking, commercially available dietary supplements of Vitamin E are alpha-tocopherol enriched compositions. As used herein, a “non-alpha-tocopherol enriched tocopherol composition” refers to a composition comprising at least 50% of any tocopherol except for alpha-tocopherol. In some examples, the non-alpha-tocopherol is gamma-tocopherol, or a metabolite thereof, beta-tocopherol, or a metabolite thereof, or delta-tocopherol or a metabolite thereof. A non-alpha tocopherol enriched tocopherol composition may comprise a mixture of tocopherols, including alpha-tocopherol, as long as the composition comprises at least 50% of a non-alpha tocopherol. As used herein, a “non-alpha-tocopherol metabolite” refers to a metabolite of a non-alpha-tocopherol, such as for example, a gamma-tocopherol metabolite, such as gamma-carboxy ethyl hydroxy chroman (gamma-CEHC); a beta-tocopherol metabolite, such as for example, beta-CEHC; or a delta-tocopherol metabolite, such as for example, delta-CEHC. These compounds are further described below.

By a “flavonoid” is meant any of a class of polyphenolic molecules (including hesperetin and derivatives thereof) based on a flavan nucleus, comprising 15 carbon atoms, arranged in three rings as C₆-C₃-C₆. Flavonoids are generally classified into subclasses by the state of oxidation and the substitution pattern at the C2-C3 unit. As used herein, the term “flavonoid” encompasses, but are not limited to, flavanones, flavonols, flavones, anthocyanidins, chalcones, dihydrochalcones, aurones, flavanols, dihydroflavanols, proanthocyanidins (flavan-3,4-diols), isoflavones and neoflavones.

As used herein, the term “flavonoids” encompasses, but is not limited to:

Chrysin (5,7-dihydroxy-2-phenyl-4H-1-benzopyran-4-one; 5,7-dihydroxyflavone, chrysidenon); daidzein (7-hydroxy-3-(4-hydroxyphenyl)-4H-1-benzopyran-4-one; 4′,7-dihydroxyisoflavone); diosmin (7-[[6-O-6-Deoxy-α-L-mannopyranosyl)-β-D-glucopyranosyl]oxy]-5-hydroxy-2-(3-hydroxy-4-methoxyphenyl)-4H-1-benzopyran-4-one); 3′,5,7-trihydroxy-4′-methoxyflavone-7-rutinoside; 5-hydroxy-2-(3-hydroxy-4-methoxyphenyl)-7-(O⁶-α-L-rhamnopyranosyl-β-D-glucopyranosyloxy)chromen-4-one; 5-hydroxy-2-(3-hydroxy-4-methoxyphenyl)-7-β-rutinosyloxy-4H-chromen-4-one; diosmetin; 7-β-rutinoside; barosmin; buchu resin; Daflon; Diosmil; Diovenor; Flebopex; Flebosmil; Flebosten; Flebotropin; Hemerven; Insuven; Tovene; Varinon; Ven-Detrex; Venex; Veno-V; Venosmine; hesperetin ((S)-2,3-dihydro-5,7-dihydroxy-2-(3-hydroxy-4-methoxyphenyl)-4H-1-benzopyran-4-one); 3′,5,7-trihydroxy-4′-methoxyflavanone; cyanidanon 4′-methyl ether 1626; hesperidin ((S)-7-[[6-O-(6-Deoxy-α-L-mannopyranosyl)-β-D-glucopyranosyl]oxy]-2,3-dihydro-5-hydroxy-2-(3-hydroxy-4-methoxyphenyl)-4H-1-benzopyran-4-one); hesperetin (7-rhamnoglucoside); cirantin; hesperetin-7-rutinoside; luteolin (2-(3,4-Dihydroxyphenyl)-5,7-dihydroxy-4H-1-benzopyran-4-one); 3′,4′,5,7-tetrahydroxyflavone; digitoflavone; cyanidenon 1470; quercetin (2-(3,4-Dihydroxyphenyl)-3,5,7-trihydroxy-4H-1-benzopyran-4-one); 3,3′,4′,5,7-pentahydroxyflavone; memtin; sophoretin; cyanidenolon 1522; rutin (3-[[6-O-(6-Deoxy-α-L-mannopyranosyl)-β-D-glucopyranosyl]oxy]-2-(3,4-dihydroxyphenyl)-5,7-dihydroxy-4H-1-benzopyran-4-one); rutoside; quercetin-3-rutinoside; 3,3′,4′,5,7-pentahydroxyflavone-3-rutinoside; melin; phytomelin; eldrin; ilixathin; sophorin; globularicitrin; paliuroside; osyritrin; osyritin; myrticolorin; violaquercitrin; Birutan; Rutabion; Rutozyd; Tanrutin; biochanin or biochanin A (5,7-dihydroxy-4′-methoxyiso-flavone); olmelin.

By “derivative of a flavonoid” is meant a compound derived from and thus non-identical to another compound. As used herein, a derivative shares at least one function with the compound from which it is derived, but differs from that compound structurally. Derivatives of flavonoids include without limitation those that differ from flavonoids due to modifications (including without limitation substitutions, additions and deletions) in a ring structure or side chain. Derivatives of hesperetin include those compounds which differ from hesperetin in structure. These structural differences can be, as non-limiting examples, by addition, substitution or re-arrangement of hydroxyl, alkyl or other group.

An “omega-3 polyunsaturated fatty acid” or “omega-3 fatty acid” is a polyunsaturated fatty acid characterized by a methylene-interrupted structure and at least two double bonds, where the first double bond is between carbons 3 and 4, relative to the methyl group. The omega nomenclature describes the position of the first double bond in the hydrocarbon relative to the methyl group. Omega-3 fatty acids are preferably in the natural “all-cis” configurations. Omega-3 polyunsaturated fatty acids include, but are not limited to 4,7,10,13,16,19-docosahexaenoic acid (DHA; C22:6n-3; indicating 22 carbons, 6 double bonds. first double bond at position 3); 7,10,13,16,19 docosapentaenoic acid (C22:5n-3; DPA), 5,8,11,14,17-eicosapentaenoic acid (EPA; C20:5n-3); 8,11,14,17-eicosatetraenoic acid (ETA;C20:4n-3); 9,12,15 octadecatrienoic acid (alpha linolenic acid, ALA; C18:3n-3), 6,9, 12,15 octadecatetraenoic acid (stearidonic acid, SDA; 18:4n-3). Compositions of the present invention may include highly enriched sources of such compounds, such as flax oil, Perilla oil (source of alpha linolenic acid), or the like. In such cases, it is preferable that such compositions contain less than about 50%, preferably less than about 25%, and more preferably less than about 10% of any omega-6 poly-unsaturated fatty acid that may be present in the mixture.

Omega-9 polyunsaturated fatty acids include, for example, 5,8,11-eicosatrienoic acid, an omega-9 fatty acid that has anti-inflammatory properties, and is produced in potentially commercial quantities by Suntory Ltd. (Osaka, JP). Other omega-fatty acids include 6,9 octadecadienoic acid and 8,11-eicosadienoic acid. U.S. Pat. No. 5,981,588, incorporated herein by reference, describes anti-allergic properties of these compounds and methods for obtaining such compounds.

As used herein “DHA” refers to the highly unsaturated fatty acid all-cis 4,7,10,13,16,19-docosahexaenoic acid and encompasses the free acid, methyl ester, ethyl ester, monoglyceride, diglyceride and triglyceride form and encompasses DHA obtainable from any source, including algal, fungal, plant, avian, fish or mammalian sources. Algal DHA is available, for example, from Martek Biosciences (Columbia, Md.) and its distributors.

By a “non-tocopherol” is meant any compound which is not a tocopherol, tocotrienol, or derivative thereof, or the like.

By “non-naturally-occurring composition” is meant a composition which is not found in this form in nature. A non-naturally-occurring composition can be derived from a naturally-occurring composition, e.g., as non-limiting examples, via purification, isolation, concentration, chemical modification (e.g., addition or removal of a chemical group), and/or, in the case of mixtures, addition or removal of ingredients or compounds. A non-naturally-occurring composition can comprise or be derived from a non-naturally-occurring combination of naturally-occurring compositions. Thus, a non-naturally-occurring composition can comprise a mixture of purified, isolated, modified and/or concentrated naturally-occurring compositions, and/or can comprise a mixture of naturally-occurring compositions in forms, concentrations, ratios and/or levels of purity not found in nature.

“Agents” or “anti-inflammatory agents” are defined herein as compounds, mixtures, or formulations of compounds which are capable of treating or ameliorating the symptoms of inflammation, such as by reducing the levels of inflammatory markers, e.g., CRP, cytokines associated with inflammation, such as members of the interleukin family, including IL-1 through 17, TNF-alpha; B61; certain cellular adhesion molecules, such as for example, e-selectin (also known as ELAM), sICAM, integrins, ICAM-1, ICAM-3, BL-CAM, LFA-2, VCAM-1, NCAM and PECAM; neopterin; serum procalcitonin; leukotriene, thromboxane, isoprostane and/or by reducing pain and/or edema associated with the inflammation. In the context of the present invention, a formulation of the invention may be referred to as an “agent.”

As used herein, “markers (or biomarkers) associated with inflammation” include, but are not limited to CRP, cytokines associated with inflammation, such as members of the interleukin family, including IL-1 through IL-17, TNF-alpha; sTNFr2; B61; certain cellular adhesion molecules, such as for example, e-selectin (also known as ELAM), sICAM, integrins, ICAM-1, ICAM-3, BL-CAM, LFA-2, VCAM-1, NCAM and PECAM; neopterin; serum procalcitonin; leukotriene, thromboxane, isoprostane, white blood cell count, and erythrocyte sedimentation rate.

By “amounts effective to reduce inflammation and/or symptoms due to inflammation” is meant that the anti-inflammatory agent or agents is administered in a sufficient dose or to achieve a final concentration sufficient for reducing inflammation, as measured by a reduction in an inflammatory marker, such as ELAM or an inflammatory cytokine, such as IL-6, or a reduction of CRP, and/or reduction of symptoms associated with inflammation, such as for example, pain and/or edema associated with inflammation. This amount includes, but is not limited to, a concentration which acts as a complete prophylaxis or treatment for a symptom of inflammation. An “effective amount” is an amount sufficient to effect beneficial or desired results. An effective amount can be administered in one or more administrations. For purposes of this invention, an effective amount of a anti-inflammatory agent is an amount that is sufficient to ameliorate, stabilize, reverse, slow or delay the progression of injury(ies) in mammalian subjects i) at risk for a disease, disorder or condition associated with inflammation, or ii) associated with, due to and/or symptoms of inflammation. Preferably, amelioration of symptoms due to inflammation can be quantified by an assay measuring, for example, reduction in CRP levels and/or reduction in inflammatory markers, such as by measuring reduction in cytokines such as, but not limited to interleukins 1-17 (IL 1-17) associated with inflammation; and TNF-alpha, as exemplified herein.

By “amelioration” is meant the prevention, reduction or palliation of a state, or improvement of the state of a subject; the amelioration of a stress is the counter-acting of the negative aspects of a stress. Amelioration includes, but does not require complete recovery or complete prevention of a stress. More specifically, amelioration may be considered to be at least about 30%, at least about 50%, at least about 70%, at least about 80%, and at least about 90% reduction in the levels of inflammatory markers associated with inflammation or an inflammatory condition or a reduction in the symptoms associated with inflammation such as for example, pain and/or edema associated with inflammation.

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms.

A “mammalian subject” or “individual” (used interchangeably herein) includes, but is not limited to, a human, a farm animal, a sport animal, and a pet.

As used herein, the term “comprising” and its cognates are used in their inclusive sense; that is, equivalent to the term “including” and its corresponding cognates.

II. Formulations

It is a discovery of the present invention that a combination of a tocopherol, particularly a non-alpha tocopherol, such as gamma-tocopherol, beta-tocopherol, and/or delta-tocopherol, and a highly unsaturated (polyunsaturated) fatty acid, such as an omega-3 or an omega-9 polyunsaturated fatty acid, such docosahexaenoic acid (DHA), is effective in treating symptoms of and reducing markers associated with inflammation.

Without relying on any particular underlying mechanism of action, it is thought that a mechanism by which the present formulations act within the mammalian subject during digestion, absorption, and systemic distribution, particularly when that subject has a concurrent inflammatory condition. Inflammation results in increased rate of production of reactive oxygen species, which attack and destroy highly unsaturated fatty acids. The distribution of the non-alpha tocopherols and their metabolites into non-lipid cellular compartments ameliorates the reactive oxygen species attack on DHA prior to its reaching its target locations in the body, where it enhances cell membrane function and has its own anti-inflammatory effects. Therefore the combination of the non-alpha tocopherol with the omega-3 fatty acid such as DHA has greater efficacy than either component alone.

A formulation may alternatively include a non-alpha tocopherol in combination with one or more flavonoids. Other components of such formulations may include a mineral, particularly a divalent cation such as magnesium, and/or a flavonoid. This section will describe exemplary components and component ratios of such formulations.

Tocopherols

Formulations of the present invention may include a pure tocopherol or a non-alpha-tocopoherol enriched tocopherol composition or mixture, namely a gamma-, delta- or beta-tocopherol, or a tocopherol derivative, or a mixture of tocopherols and/or tocotrienols that is enriched in a non-alpha tocopherol (i.e., where alpha-tocopherol comprises less than 25%, and preferably less than 10% of tocopherols present in the formulation). In particular, non-alpha tocopherols that are effective in anti-inflammatory compositions of the present invention include gamma, delta, and beta tocopherol. Other tocopherol derivatives, in accordance with the present invention, include known metabolites of tocopherols, for example, alpha- and gamma-tocopherol metabolites 2,5,7,8-tetramethyl-2-(2′-carboxyethyl)-6-hydroxychroman and 2,7,8-trimethyl-2-(2′-carboxyethyl)-6-hydroxychroman (gamma-CEHC). Additional gamma-tocopherol metabolites and derivatives are known in the art or are described, for example, in U.S. Pat. Nos. 6,048,981 and 6,083,982, both of which are incorporated herein by reference.

Other non-alpha tocopherols useful in formulations of the invention may be determined empirically in accordance with the teachings of the present invention, with reference to the cellular anti-inflammatory assay described herein.

Tocopherols are chemical entities which, in general, contain a 6-chromanol ring structure and a side chain at the 2-position. As mentioned above, prototypical non-alpha tocopherols include beta-, delta- and gamma-tocopherol. The tocopherols have the general formula:

Tocopherols:

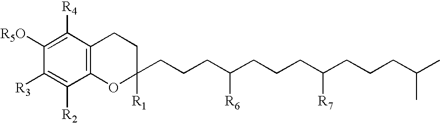

R1=CH3 with S or R configuration

R6=CH3 with S or R configuration

R7=CH3 with S or R configuration

R5=H or CH3 or acetate or succinate

| | R2 | R3 | R4 |
|---|---|---|---|
| Alpha | CH3 | CH3 | CH3 |
| Gamma | CH3 | CH3 | H |
| Beta | CH3 | H | CH3 |
| Delta | CH3 | H | H |

As discussed herein, tocopherols for use in the present invention are non-alpha tocopherols. In general, supplements that contain “Vitamin E” are understood to be composed predominantly of alpha-tocopherol. Tocopherols and their derivatives can vary by the number and position of alkyl groups, double bonds and other substituents and variations on the ring and side chain. In preferred embodiments, the tocopherol component of formulations of the present invention is predominantly a gamma-tocopherol, a beta-tocopherol, or a delta-tocopherol. In another preferred embodiment, the tocopherol component is made up of “mixed tocopherols,” such as those that are isolated from natural sources, with the proviso that such mixed tocopherol component will preferably contain or be supplemented to contain less than about 25% or more preferably less than 10% alpha tocopherol. Tocopherols may be obtained from a variety of sources, including Cargill, Incorporated (Minnetonka, Minn.), which processes a 95% pure gamma-tocopherol product, or Cognis Nutrition and Health (Cincinnati, Ohio), which markets a 92% pure gamma-tocopherol product.

Tocopherol derivatives may be constructed according to methods known in the chemical arts. In this context, an “alkyl” is a cyclic, branched or straight chain chemical group containing only carbon and hydrogen, such as methyl, butyl and octyl. Alkyl groups can be either unsubstituted or substituted with one or more substituents, e.g., halogen, alkoxy, acyloxy, amino, hydroxyl, mercapto, carboxy, or benzyl. Alkyl groups can be saturated or unsaturated at one or several positions. Typically alkyl groups will comprise 1 to 8 carbons, preferably 1 to 6, and more preferably 1 to 4 carbon atoms. Additional tocopherols can be constructed by conjugation to the ring structure or side chain of various other moieties, such as those containing oxygen, nitrogen, sulfur and/or phosphorus. Tocopherol derivatives can also be made, as known in the art, by modifying the length of the side chain from that found in prototypical tocopherols such as alpha-, beta-, delta- and gamma-tocopherol. Tocopherols can also vary in stereochemistry and saturation of bonds in the ring structure and side chain. Additional tocopherol derivatives, including prodrugs, can be made by conjugation of sugars or other moieties to the side chain or ring structure; these can serve any of a number of functions, including increasing solubility and increasing functional activity of the tocopherol. Thus, as is understood in the art, the invention encompasses the use of tocopherol derivatives in which substitutions, additions and other alterations have been made in the 6-chromanol ring and/or side chain, with the proviso that the derivatives maintain at least one functional activity of a tocopherol, such as antioxidant activity or ability to counteract sterility in animals. More preferably, by way of guidance, tocopherol derivatives useful in the invention will have CRP-lowering activity, such as in a cellular assay of CRP production, as described in Example 1, herein.

An exemplary mixed tocopherol composition can be obtained, for example from Cargill Incorporated [Minnetonka, Minn.], and contains 62% gamma tocopherol, 28% delta tocopherol, 8% alpha tocopherol and less than 2% beta tocopherol. Additional mixed tocopherols from natural and transgenic sources are described, for example in PCT Publication WO 00/10380, incorporated herein by reference. Preferably, such mixed tocopherols will consist of less than 25%, and more preferably less than 10% alpha-tocopherol. Such mixed tocopherols may contain tocotrienols or other tocopherol-like derivatives in addition to the tocopherols mentioned above. Soybean oil is a particularly preferred natural source of mixed tocopherols of the invention; other preferred sources may include palm oil, corn oil, whole grain corn, safflower oil, rapeseed oil, whole wheat flour, or castor bean oil. Cargill and other commodities processors are sources for many of these materials. Preferred transgenic sources, as described in PCT Publication WO 00/10380, incorporated herein by reference, include soybean oil, oil palm oil, rapeseed oil, corn oil, and whole grain corn. Other natural and transgenic, enriched or otherwise artificially engineered sources will be readily apparent to the practitioner, with the guidance of the compositional guidance provided herein.

In further embodiments, the tocopherol component may be a metabolite of gamma-, delta- or beta-tocopherol, either in its administered or in vivo transformed form. One exemplary metabolite of gamma tocopherol is gamma-carboxy ethyl hydroxy chroman (gamma-CEHC), such as is further described by U.S. Pat. No. 6,083,982, incorporated herein by reference. The present invention also provides compositions comprising a gamma-tocopherol metabolite, a beta-tocopherol metabolite, and/or a delta-tocopherol metabolite, such as are well known in the art.

In the body of a subject, gamma-tocopherol breaks down into metabolites, including for example, the metabolites described in U.S. Pat. Nos. 6,150,402; 6,083,982; 6,048,891; and 6,242,479, specifically incorporated herein in their entireties. In particular, the present invention encompasses the use of gamma-tocopherol enriched tocopherol compositions that further comprise a gamma-tocopherol metabolite such as gamma-CEHC, racemic gamma-CEHC and (S) gamma-CEHC.

In the body of a subject, beta-tocopherol breaks down into metabolites. In particular, the present invention encompasses the use of compositions that comprise a beta-tocopherol metabolite such as 2,5,8-trimethyl-2-(2-carboxyethyl)-6-hydroxychroman (beta-CEHC). The present invention encompasses the use of compositions that comprise a beta-tocopherol metabolite such as beta-CEHC, racemic beta-CEHC and (S) beta-CEHC.

In the body of a subject, delta-tocopherol breaks down into metabolites. In particular, the present invention encompasses the use of compositions that comprise a delta-tocopherol metabolite such as delta-CEHC, racemic delta-CEHC and (S) delta-CEHC.

Derivatives of these compounds include, but are not limited to structural derivatives, as described above, as well as salts, including but not limited to succinate, nicotinate, allophanate, acetate, and phosphate salts of the tocopherols described herein. Salts also include pharmaceutically acceptable salts. Derivatives also include quinone derivatives and prodrug forms of tocopherols, such as those described in U.S. Pat. No. 5,114,957. Additional tocopherols and derivatives thereof are described in, e.g., U.S. Pat. Nos. 5,606,080 and 5,235,073. Preparation of various tocopherols are described in, e.g., U.S. Pat. Nos. 5,504,220, 4,978,617, and 4,977,282. Various tocopherols are commercially available, for example from Sigma Chemical Co., St. Louis, Mo.

Polyunsaturated Fatty Acids

Exemplary highly unsaturated fatty acids that may be used in the formulations and methods of the invention are preferably omega-3 fatty acids, such as, for example, all-cis 4,7,10,13,16,19-docosahexaenoic acid (DHA; C22:6n-3); 5,8,11,14,17-eicosapentaenoic acid (EPA; C20:5n-3); or 9,12,15-octadecatrienoic acid (C18:3n-3). Alternatively, the highly unsaturated fatty acid may be an omega-9 fatty acid such as 5,8,11-eicosatrienoic acid (C20:3n-9, also known as “Mead acid”), or other poly-unsaturated fatty acids known in the art.

Polyunsaturated fatty acids are commercially available from a number of vendors. DHA can be obtained, for example, from Martek Biosciences Corporation (Columbia, Md.). Martek provides a microalgae-derived product, a 40% DHA product marketed as “NEUROMINS.” U.S. Pat. Nos. 5,492,938 and 5,407,957, incorporated herein by reference, describe methods of producing DHA from microalgae. DHA from other sources, including cold-water ocean fish, sea mammals, arid range-fed poultry, as well as other omega-3 fatty acids, are also commercially available from sources known in the art.

Similarly omega-9 polyunsaturated fatty acids have been characterized as anti-allergy compounds in U.S. Pat. No. 5,981,588, incorporated herein by reference, and are available from Suntory Ltd. (Osaka, Japan).

Other highly unsaturated fatty acids are known in the art, for example U.S. Pat. No. 6,376,688, incorporated herein by reference, describes certain anti-malarial, neutrophil stimulatory polyunsaturated fatty acids characterized by their enhanced stability in vivo, by virtue of exhibiting slower metabolic turnover, for example, 8-hydroperoxy-5Z,9E,11Z,14Z-eicosatetraenoic acid.

Derivatives of the aforementioned polyunsaturated fatty acids are also suitable for use in the invention, for example, esters of DHA, glycerides of DHA, and the like, such as described in U.S. Pat. No. 5,436,269, incorporated herein by reference.

Flavonoid Component

In another embodiment, the formulation may include at least one flavonoid, such as is defined in the “Definitions” section herein. In some embodiments, the compositions comprise at least two such flavonoids. In yet other preferred embodiments, the flavonoids include chrysin, diosmin, hesperetin, luteolin, rutin, or quercetin. In additional embodiments, the flavonoids present in the formulation are hesperetin and quercetin, singly, or more preferably, in combination. Thus, in some embodiments of the present invention, compositions comprise gamma-tocopherol, hesperetin, quercetin and DHA. Ranges and approximate dosages are described below.

Flavonoids comprise a class of polyphenolic substances based on a flavan nucleus, generally comprising 15 carbon atoms, arranged in three rings as C₆-C₃-C₆. There are a number of chemical variations of the flavonoids, such as, the state of oxidation of the bond between the C2-C3 position and the degree of hydroxylation, methoxylation or glycosylation (or other substituent moieties) in the A, B and C rings and the presence or absence of a carbonyl at position 4. Flavonoids include, but are not limited to, members of the following subclasses: chalcone, dihydrochalcone, flavanone, flavonol, dihydroflavonol, flavone, flavanol, isoflavone, neoflavone, aurone, anthocyanidin, proanthocyanidin (flavan-3,4-diol) and isoflavane.

Flavanones contain an asymmetric carbon atom at the 2-position and flavanones include, but are not limited to, narigenin, naringin, eriodictyol, hesperetin and hesperidin. Dihydroflavonols include, but are not limited to, taxifolin (dihydroquercetin). Flavones include, but are not limited to, chrysin, diosmin, luetolin, apigenin, tangeritin and nobiletin. Flavonols include, but are not limited to, kampferol, quercetin and rutin. Flavanes include, but are not limited to, catechin and epi-gallocatechin-gallate. Isoflavones include, but are not limited to, biochanin, daidzein, glycitein and genistein.

In some embodiments, compositions comprise a flavanone. In further embodiments, compositions comprise the flavanone hesperetin. In other embodiments, compositions comprise flavonols, such as, quercetin. In yet further embodiments, the compositions comprise a isoflavone. In other embodiments, the compositions comprise a flavone. In further embodiments, the compositions comprise a flavonol.

Hesperetin and hesperidin are flavonoids found in citrus, such as lemons, grapefruits, tangerines and oranges, and may be extracted from the peel of citrus or synthesized according to the process described by Shinoda, Kawagoye, C.A. 23:2957 (1929); Zemplen, Bognar, Ber., 75,1043 (1943) and Seka, Prosche, Monatsh., 69, 284 (1936). Hesperetin may also be prepared by the hydrolysis of hesperidin (see, for example, U.S. Pat. No. 4,150,038).

Daidzein is a flavonoid isolated from red clover (Wong (1962) J. Sci. Food Agr. 13:304) and from the mold Micromonospora halophytica (Ganguly et al. Chem. & Ind. (London) 197, 201. Additional descriptions of isolation of daidzein from various plant products can be found in Hosny et al. (1999) J. Nat. Prod. 62: 853-858 and Walz (1931) Ann. 489:118. Synthesis of daidzein is described in Farkas et al. (1959) Ber. 92:819. Daidzein is an inactive analog of the tyrosine kinase inhibitor genistein (Sargeant et al. (1993) J. Biol. Chem. 268:18151). Daidzein is also a phytoestrogen, recently suggested to play a role in preventing special types of cancer. See, for example, Sathyamoorthy et al. (1994) Cancer Res. 54:957; Zhou et al. (1999) J. Nutr. 129: 1628-1635 and Coward et al. (1993) J. Agric. Food Chem. 41:1961. Daidzein also has anti-estrogen properties (Anderson et al. (1998) Baillieres Clin. Endocrinol. Metab. 12: 543-557). Daidzein also acts as an anti-oxidant, inhibiting lipid peroxidation. Arora et al. (1998) Arch. Biochem. Biophys. 356: 133-41; and Hodgson et al. (1999) Atherosclerosis 145: 167-72.

Biochanin A can be isolated from red clover (Pope et al. (1953) Chem. & Ind. (London) 1092 and Wong (1962) J. Sci. Food. Agr. 13:304) and its structure is described by Bose et al. (1950) J. Sci. Ind. Res. 9B:25. Biochanin A has some anti-cancer properties. Lyn-Cook et al. (1999) Cancer Lett. 142: 111-119; Hammons et al. (1999) Nutr. Cancer 33: 46-52; Yin et al. (1999) Thyroid 9: 369-376. Biochanin A also has anti-oxidant properties, including the ability to inhibit lipid peroxidation. Toda et al. (1999) Phytother. Res. 13: 163-165.

Flavonoids isolated and purified from natural sources or chemically synthesized may be used in the invention. Methods to isolate and identify flavonoids have been described, for example, in Markham et al. (1998) pp. 1-33, in Flavonoids in Health and Disease, Rice-Evans and Packer, eds. Marcel Dekker, Inc. Many flavonoids are commercially available from sources such as Funakoshi Co., Ltd. (Tokyo), Sigma Chemical Co. (St. Louis, Mo.) and Aldrich Chemical Co. (Milwaukee, Wis.). Generally, hesperetin, hesperidin, quercetin, diosmin, daidzein, chyrsin, luteolin, biochanin and rutin are available from commercial sources.

Also suitable in the present invention are derivatives of flavonoids. For example, a derivative of a flavonoid differs from the flavonoid in structure. These differences can be, as non-limiting examples, by addition, substitution or re-arrangement of hydroxyl, alkyl or other group. As a non-limiting example, a flavonoid derivative can have additional alkyl groups attached. In addition, flavonoid derivatives include compounds which have been conjugated to another chemical moiety, such as a sugar or other carbohydrate. Other suitable moieties contain oxygen, nitrogen, sulfur, and/or phosphorus. Derivatives of flavonoids can be produced, for example, to improve its solubility, reduce its odor or taste, or to ensure that the compound is free of toxicity. A flavonoid can also be conjugated to another moiety to form a prodrug. In a prodrug, a flavonoid is conjugated to a chemical moiety which, for example, aids in delivery of the flavonoid to the site of activity (e.g., a particular tissue within the body). This chemical moiety can be optionally cleaved off (e.g., enzymatically) at that site.

Hesperetin derivatives are described in, for example, Esaki et al. (1994) Biosci. Biotechnol. Biochem. 58:1479-1485; Scambia et al. (1990) Anticancer Drugs 1:45-48; Bjeldanes et al. (1977) Science 197:577-578; Honohan et al. (1976) J. Agric. Food Chem. 24:906-911; and Brown et al. (1978) J. Agric. Food Chem. 26:1418-1422.

While differing from the flavonoid in structure, derivatives of the flavonoid will retain at least one activity of the flavonoid. Generally it is anticipated that such derivatives will exhibit some level of anti-inflammatory activity, as measured, for example in the ELAM assay detailed in Example 1, herein. For hesperetin and hesperetin derivatives these activities may include anti-oxidant and anti-free radical activity (Saija et al. (1995) Free Radic. Biol. Med. 19:481-486),

Derivatives of diosmin include diosmin heptakis (hydrogensulfate) aluminum complex, and diosmin octakis (hydrogen sulfate) aluminum complex, as described in U.S. Pat. Nos. 5,296,469; and 4,894,449. Another derivative of diosmin is its aglycone form, diosmetin, 5,7-dihydroxy-2-(3-hydroxy-4-methoxypenyl)-4H-1-benzopyran-4-one. See The Merck Index (1989), Eleventh Edition, p. 520, and references cited therein. Derivatives of diosmin also include salts thereof. A synthetic diosmin derivative, LEW-10, is described in Azize et al. (1992) Chem. Phys. Lipids 63:169-77.

While differing from diosmin in structure, diosmin derivatives will retain at least one activity of diosmin. For example, diosmin is known to exhibit free radical scavenger activity (Dumon et al. (1994) Ann. Biol. Clin. 52: 265-270) and is an antilipoperoxidant (Feneix-Clerc et al. (1994) Ann. Biol. Clin. 52:171-177). The combination of diosmin and hesperidin, known as DAFLON™ 500, has been alleged to exhibit anti-inflammatory, anti-free radical, venotonic and vasculoprotective activities, in addition to attenuating reperfusion injury. Guillot et al. (1998) Pancreas 17:301-308; Amiel et al. (1998) Ann. cardiol. Angeiol. 47:185-188; Nolte et al. (1997) Int. J. Microcirc. Clin. Exp. 17 (suppl. 1): 6-10; Delbarre et al. (1995) Int. J. Microcirc. Clin. Exp. 15 (suppl. 1): 27-33; Bouskela et al. (1995) Int. J. Microcirc. Clin. Exp. 15 (suppl. 1):22-6; and Friesenecker et al. (1995) Int. J. Microcirc. Clin. Exp. 15 (suppl. 1):17-21. The combination of diosmin and hesperidin is also allegedly useful for treating hemorrhoids. U.S. Pat. No. 5,858,371. A diosmin derivative retains at least one of these activities.

Derivatives of daidzein, biochanin A and other compounds described herein include compounds which are chemically and/or structurally similar, but non-identical to such compounds, and which share at least one function of those compounds. Numerous derivatives of daidzein are known in the art. These include daidzein 7-glucoside, or daidzin; and the aglucon of daidzein. Glycosylated and methoxylated derivatives of daidzein are described in Arora et al. (1998). Chlorinated derivatives of daidzein are described in Boersma et al. (1999) Arch. Biochem. Biophys. 368: 265-275. Additional derivatives are described in Lapcik et al. (1997) Steroids 62: 315-320; Joannou et al. (1995) J. Steroid. Biochem. Mol. Biol. 54: 167-184; Keung (1993) Alcohol Clin. Exp. Res. 17: 1254-1260; Smit et al. (1992) J. Biol. Chem. 267: 310-318; Shao et al. (1980) Yao Hsueh Hsueh Pao 15: 538-547 and King et al. (1998) Am. J. Clin. Nutr. 68:1496S-1499S. Numerous derivatives of biochanin A are also described in the art, in, for example, chlorinated derivatives described in Boersma et al. (1999).

Minerals

Compositions of the present invention may also include a mineral supplement, such as magnesium. Other mineral supplements may be used, for example copper, zinc, selenium, molybdenum, manganese, chromium, iodine, iron and combinations thereof. In formulations of the present invention, divalent ions, such as calcium and magnesium, zinc, and manganese are preferred.

Other Ingredients

It is understood that formulations may include other ingredients that may augment or enhance anti-inflammatory activity, stability or other desirable feature of the formulation. One such ingredient is alpha-lipoic acid (1,2-dithia-cyclopentane-3-valeric acid), the pure R-form of which has been described for use in treating diabetes in U.S. Pat. No. 5,693,664 and in combination with vitamin E in U.S. Pat. No. 5,569,670, both of which patents are incorporated herein by reference. Other beneficial ingredients will be readily apparent to persons skilled in the art.

Excipients and Preparations

In further embodiments, formulations of the present invention comprise an excipient suitable for use in dietary or nutritional supplements. For example, in studies carried out in support of the present invention tocopherol-containing formulations were prepared in high oleic sunflower oil (A. C. Humko (TRISUN 80; Cordova, Tenn.)). Other acceptable nutritional excipients are well known in the art, and may include, without limitation, binders, coatings, disintegrants, and hydrocolloids, which may be used advantageously to provide desired properties. Such products may be obtained form a variety of sources, for example, FMC Corporation (Philadelphia, Pa.). Formulations may also comprise an excipient suitable for pharmaceutical uses; such excipients are well known in the art (See, e.g., Remington's Pharmaceutical Sciences).

In another embodiment, formulations of the invention will be incorporated into a daily “vitamin” regimen. For example, the components can incorporated into standard multi-vitamins, or may be included as additional capsules in a multi-vitamin supplement package which includes a variety of dietary supplements or “pills” in a pre-wrapped format, such as in a sealed cellophane packet containing pre-defined dosage(s). Alternatively, the various components of the formulation can be separately bottled and sold, or suggested to be purchased, in combination.

The compositions, as described above, can be prepared as a medicinal preparation (such as an aqueous solution for injection) or in various other media, such as foods for humans or animals, including medical foods and dietary supplements. A “medical food” is a product that is intended for the specific dietary management of a disease or condition for which distinctive nutritional requirements exist. By way of example, but not limitation, medical foods may include vitamin and mineral formulations fed through a feeding tube to cancer or burn victims (referred to as enteral administration or gavage administration). A “dietary supplement” shall mean a product that is intended to supplement the human diet and is typically provided in the form of a pill, capsule, tablet or like formulation. By way of example, but not limitation, a dietary supplement may include one or more of the following ingredients: vitamins, minerals, herbs, botanicals, amino acids, dietary substances intended to supplement the diet by increasing total dietary intake, and concentrates, metabolites, constituents, extracts or combinations of any of the foregoing. Dietary supplements may also be incorporated into food stuffs, such as functional foods designed to promote tissue health or to prevent Inflammation.

Generally, the route(s) of administration useful in a particular application are apparent to one of skill in the art. Routes of administration include, but are not limited to, oral, topical, dermal, transdermal, transmucosal, epidermal, parenteral, gastrointestinal. Specific methods for these routes of administration are known in the art.

Formulations may be conveniently packaged in a, in accordance with one preferred aspect of the invention, according to methods well known in the art. By way of example, but not limitation, such oral forms may include be prepared as solid dosage forms, sustained and controlled release forms, liquids, or semi-solids

For oral administration, formulations of the invention may be administered in nutritionally accepted vehicles for oral ingestion, such as, capsules, tablets, or pills, soft gel caps, powders, solutions, dispersions, or liquids. In preparing the compositions in oral dosage form, any of the usual media may be employed. For oral liquid preparations (e.g., suspensions, elixirs, and solutions), media containing, for example, water, oils, alcohols, flavoring agents, preservatives, coloring agents and the like may be used. Carriers such as starches, sugars, diluents, granulating agents, lubricants, binders, disintegrating agents, and the like may be used to prepare oral solids (e.g., powders, capsules, pills, tablets, and lozenges). Controlled release forms may also be used. A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethylcellulose), lubricant, inert diluent, preservative, disintegrant (e.g. sodium starch glycollate, cross-linked povidone, cross-linked sodium carboxymethylcellulose) surface-active or dispersing agent. Molded tablets may be made by moulding in a suitable machine a mixture of the powdered compound moistened with an inert liquid diluent. The tablets may optionally be coated or scored and may be formulated so as to provide controlled release of the active ingredients therein using, for example, hydroxypropylmethylcellulose in varying proportions to provide the desired release profile. Soft gelcaps are particularly preferred in containing lipophilic substances, such as tocopherols and polyunsaturated fatty acids. Methods for preparing gelcaps are well known in the art.

Also, the subject formulations may be compounded with other physiologically acceptable materials which can be ingested including, but not limited to, foods, including, but not limited to, food bars, beverages, powders, cereals, cooked foods, food additives and candies. When the composition is incorporated into various media such as foods, it may simply be orally ingested. The food can be a dietary supplement (such as a snack or wellness dietary supplement) or, especially for animals, comprise the nutritional bulk (e.g., when incorporated into the primary animal feed).

For rectal administration, the subject compositions may be provided as suppositories, as solutions for enemas, or other convenient application. Suppositories may have a suitable base comprising, for example, cocoa butter or a salicylate. Formulations for vaginal administration may be presented as pessaries, tampons, creams, gels, pastes, foams or spray formulations containing in addition to the active ingredient such carriers as are known in the art to be appropriate.

Otherwise, the subject compositions may be administered intravascularly, arterially or venous, subcutaneously, intraperitoneally, intraorganally, intramuscularly, by dermal patch, or the like.

The subject compositions may be administered parenterally including intravascularly, arterially or venous, subcutaneously, intradermally, intraperitoneally, intraorganally, intramuscularly, or the like.

Formulations for parenteral administration include aqueous and non-aqueous isotonic sterile injection solutions which may contain buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

For topical administration, the subject compositions may be provided as a wide variety of product types including, but are not limited to, lotions, creams, gels, sticks, sprays, ointments and pastes. These product types may comprise several types of formulations including, but not limited to solutions, emulsions, gels, solids, and liposomes.

Compositions useful for topical administration of the compositions of the present invention formulated as solutions typically include a pharmaceutically-acceptable aqueous or organic solvent. The terms “pharmaceutically-acceptable organic solvent” refer to a solvent which is capable of having a formulation of the invention, or specified components thereof, dissolved therein, and of possessing acceptable safety properties (e.g., irritation and sensitization characteristics). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, isopropanol, butanetriol, sorbitol esters, 1,2,6-hexanetriol, ethanol, isopropanol, butanediol, and mixtures thereof. Other useful forms for topical administration include emollients, ointments, emulsions, lotions, creams and the like. Methods for preparing such preparations are well known in the art.

Liposomal formulations are also useful for the compositions of the present invention. Such compositions can be prepared by combining a formulation prepared in accordance with the present invention, with a phospholipid, such as dipalmitoylphosphatidyl choline, cholesterol and water according to known methods, for example, as described in Mezei et al. (1982) J. Pharm. Pharmacol. 34:473-474, or a modification thereof. Epidermal lipids of suitable composition for forming liposomes may be substituted for the phospholipid. The liposome preparation is then incorporated into one of the above topical formulations (for example, a gel or an oil-in-water emulsion) in order to produce the liposomal formulation. Other compositions and pharmaceutical uses of topically applied liposomes are described for, example, in Mezei (1985) Topics in Pharmaceutical Sciences, Breimer et al. eds., Elsevier Science, New York, N.Y., pp. 345-358.

The above-mentioned compositions and methods of administration are meant to describe but not limit the methods and compositions of the present invention. The methods of producing various compositions and devices are within the ability of one skilled in the art and are not described in detail here.

Ranges and Ratios of Components in Formulations of the Invention

The amount of the composition ingested, consumed or otherwise administered will depend on the desired final concentration. Typically, the amount of a single administration of the formulation of the invention can be about 0.1 to about 1000 mg per kg body weight, or about 0.5 to about 10,000 mg per day. Any of these doses can be further subdivided into separate administrations, and multiple dosages can be given to any individual patient. A typical dosage for vitamin E (alpha tocopherol) administration is 100-1000 mg/day for an adult human. However, various different dosages are described in scientific publications; see, for example, Ng et al. (1999) Food Chem. Toxicol. 37: 503-8; Ko et al. (1999) Arch. Phys. Med. Rehabil. 80: 964-7; Chen et al. (1999) Prostaglandins Other Lipid Mediat. 57: 99-111; and Thabrew et al. (1999) Ann. Clin. Biochem. 36: 216-20.

To determine the optimum concentration for any application, conventional techniques may be employed. Thus, for in vitro and ex vivo use, a variety of concentrations may be used and various assays employed to determine the degree of inflammation.

Generally, amounts of each component of the formulation are administered in a dietary supplement form will be within a range of doses that would be found in the diets of humans. Higher amounts may be used in regimens that are administered or overseen by clinical professionals. While multi-component dietary supplements generally provide about 100-200% of the Dietary Reference Intake for vitamin E, which is currently set at 15 mg/day, higher dosages of tocopherols may be administered, under appropriate regulatory and toxicological guidelines.

Formulations of the present invention may include a non-alpha tocopherol, as defined above, such as gamma tocopherol, in the range of 10 milligrams (mg) to 10,000 mg, more generally in the range of 20 mg to 1000 mg. Preferably, dosages of between about 10 mg and 500 mg, particularly between about 100 mg and 300 mg, will be ingested daily. Dosages of other non-alpha tocopherols may be determined empirically, with reference to gamma tocopherol. For example, in studies carried out in support of the present invention, subjects self-administered 300 mg of a gamma-enriched tocopherol mixture daily, in conjunction with other components of the formulation of the present invention. Other tocopherols may be substituted in such a regimen, and overall efficacy compared to that of gamma-tocopherol in relieving inflammatory symptoms or markers. More generally, it is anticipated that tocopherols that are preferred for use in the present invention will exhibit CRP-lowering activity in vitro, for example, activity comparable to that of gamma-tocopherol in a CRP lowering assay, such as the cell assay detailed in Example 1A herein.

By way of example, according to the present invention, the tocopherol component of an effective formulation may include 300 mg of “mixed tocopherols” available as a commodity, for example, as a combination of 200 mg of gamma-tocopherol, and the remainder a mixture of delta and/or beta tocopherol, with less than 25%, and preferably less than 10% alpha-tocopherol present in the mixture.

According to a further aspect of the invention, an omega-3 polyunsaturated fatty acid, such as docosahexaenoic acid (DHA), is added to the tocopherol to produce an effective formulation for reducing inflammatory symptoms, such as reducing one or more inflammatory biomarkers. This component can be incorporated with the tocopherol(s) in a single administration, or can be given separately, in a regimen designed to provide the desired level.

The average dietary intake of DHA (10-60 mg/day) in the American diet is relatively low in comparison to intake in countries where fish or fish products comprise higher percentages of the diet. Toxicological studies have demonstrated that 50× these levels (e.g., 3.6 gm DHA per day) can be ingested by humans with no apparent toxicities (Grimsgaard S, et al. Am J Clin Nutr 66:649-659, 1997). Generally, ranges of about 10-10,000 mg, or more specifically, about 50-2000 mg, or 100-1000 mg will-be preferred. In studies carried out in support of the present invention, subjects ingested approximately 800 mg DHA daily, or just over 10× an average American dietary amount, with no apparent adverse effects. Appropriate dosages of other polyunsaturated fatty acids can be estimated with reference to this study, based on known safe ingestion levels, or may be determined empirically, with the guidance provided herein.

Flavonoids may be added to formulations of the present invention, either in combination or in separate administered doses, as described herein. There are a wide variety of flavonoids present in foods commonly ingested by humans. Particularly rich sources of flavonoids include onions, apples, tea and cabbage. While there are no DRI or UL (upper limit) values established for flavonoids, American dietary intakes are estimated at below 20 mg/day. In studies carried out in support of the present invention, subjects ingested a combination of flavonoids amounting to 100 mg total supplemental flavonoids, specifically quercetin and hesperetin. Other flavonoids can be substituted in this regimen, as described above. More generally, flavonoids will be added in the range of 10-1000 mg, 20-800 mg, 50-500 mg, 50-300 mg, 100-200 mg, less than 1000 mg, less than 800 mg, less than 500 mg, less than 300 mg, less than 200 mg, greater than 10 mg, greater than 20 mg, greater than 30 mg, greater than 50 mg, greater than 100 mg.

A mineral, preferably a divalent ion such as magnesium, may be added to the tocopherol and polyunsaturated fatty acid components mentioned above. Magnesium dietary intake is generally in the range of 50-500 mg/day. Leafy green vegetables and whole grains are particularly robust dietary sources of magnesium. The United States adult DRI for magnesium is 400 mg/day; however, most adults (especially women) ingest far less. By way of example, a formulation containing 100 mg magnesium would provide 25% of the DRI. Accordingly, formulations of the invention may include magnesium in the range of 10-1000 mg, 20-800 mg, 50-400 mg, 50-300 mg, 100-200 mg, less than 1000 mg, less than 800 mg, less than 400 mg, less than 250 mg, less than 200 mg, greater than 10 mg, greater than 20 mg, greater than 30 mg, greater than 50 mg, greater than 100 mg. Other minerals can be substituted with reference to their DRIs and Upper Limits (Reference: Food and Nutrition Board, Institute of Medicine, Washington, D.C.), since toxicity may occur at very high doses of certain minerals.

In some embodiments, compositions are administered in one dosing of a single formulation and in other embodiments, compositions are administered in multiple dosing of a single formulation. In some embodiments, all components of a composition are administered together in a single formulation, that is, all components are present in a single formulation and in other embodiments, all components of a compositions are administered separately in two formulations or multiple formulations, such that all components are administered to a subject within a specified time period. In some embodiments, the time period is between about 3 hours to about 6 hours. In other embodiments, the time period is between about 6 hours and 12 hours. In additional embodiments, the time period is between about 12 hours and 24 hours. In yet further embodiments, the time period is between about 24 hours and 48 hours. The administration of separate formulations can be simultaneous or staged throughout a specified time period, such that all ingredients are administered within the specified time period.

For example, for administration of the following components: 300 mg of mixed tocopherols (180 mg gamma-tocopherol; 30 mg alpha-tocopherol; and 90 mg delta-tocopherol); 100 mg hesperetin; 200 mg quercetin; and 800 mg docosahexaenoate (DHA) per day per mammalian subject, the ingredients are administered as a) one composition comprising all components in a single dosing; b) one composition containing less than the total of all components in two or multiple dosings within a specified time period, such as for example two dosings per day per mammalian subject of formulations comprising 150 mg of mixed tocopherols (90 mg gamma-tocopherol; 15 mg alpha-tocopherol; and 45 mg delta-tocopherol); 50 mg hesperetin; 100 mg quercetin; and 400 mg docosahexaenoate (DHA); c) two or multiple compositions administered in one dose per day per mammalian subject, such as for example, 300 mg of mixed tocopherols (180 mg gamma-tocopherol; 30 mg alpha-tocopherol; and 90 mg delta-tocopherol) administered in one composition once a day along with 300 mg of flavonoids (100 mg hesperetin; 200 mg quercetin) administered in one composition once a day along with 800 mg DHA administered in one composition once per day; d) two or multiple compositions administered in a staged manner throughout the day, such as for example, 300 mg of mixed tocopherols (180 mg gamma-tocopherol; 30 mg alpha-tocopherol; and 90 mg delta-tocopherol) administered in one composition once a day along with 300 mg of flavonoids (100 mg hesperetin; 200 mg quercetin) administered in one composition once per day along with a composition comprising 200 mg DHA administered 4 times staged throughout the day; or e) each component in its own composition administered either once a day if the composition comprises the total desired amount of the component to be administered per day or multiple times a day if the composition comprises less than the total desired amount of ingredient to be administered per day with administrations throughout the day up to the total amount of components to be administered.

Illustrative examples of ranges of components in compositions include:

gamma-tocopherol or a gamma-tocopherol enriched tocopherol composition or beta-tocopherol or a beta-tocopherol enriched composition or delta-tocopherol or a delta-tocopherol enriched composition or a gamma-, beta-, or delta-tocopherol metabolite, ranging from in the lower limit at least about 10 mg, at least about 50 mg, at least about 100 mg, at least about 150 mg, at least about 200 mg, at least about 250 mg, at least about 300 mg, at least about 350 mg, or at least about 400 mg per mammalian subject per day and ranging from in the upper limit not greater than about 2000 mg, not greater than about 1500 mg, not greater than about 1250 mg, not greater than about 1000 mg, not greater than about 750 mg, not greater than about 500 mg per mammalian subject per day, wherein the lower limit and the upper limit are selected independently and in some embodiments the range of gamma-tocopherol or a gamma-tocopherol enriched tocopherol composition or beta-tocopherol or a beta-tocopherol enriched composition or delta-tocopherol or a delta-tocopherol enriched composition or a gamma-, beta-, or delta-tocopherol metabolite is from about 10 to about 1000 mg, or from about 50 to about 600 mg, or from about 100 to about 400 mg per mammalian subject per day;

hesperetin or quercetin, ranging from in the lower limit, at least about 10 mg, at least about 25 mg, at least about 50 mg, at least about 75 mg, at least about 100 mg at least about 125 mg, at least about 150 mg, at least about 200 mg, or at least about 250 mg per mammalian subject per day and ranging from in the upper limit not greater than about 1000 mg, not greater than about 750 mg, not greater than about 500 mg, not greater than about 475 mg, not greater than about 450 mg, not greater than about 425 mg, not greater than about 400 mg, not greater than about 375 mg, not greater than about 350 mg, not greater than about 325 mg, or not greater than about 300 mg wherein the lower limit and the upper limit are selected independently and in some embodiments the range of hesperetin or quercetin is from about 10 to about 500 mg, or from about 25 to about 200 mg, or from about 50 to about 100 mg per mammalian subject per day; and

DHA ranging from in the lower limit at least about 25 mg, at least about 50 mg, at least about 75 mg, at least about 100 mg, at least about, 125 mg, at least about 150 mg, at least about 175 mg, at least about 200 mg, at least about 250 mg, at least about 275 mg, at least about 300 mg, at least about 325 mg, at least about 350 mg, or at least about 400 mg per mammalian subject per day and ranging from in the upper limit not greater than about 1500 mg, not greater than about 1250 mg, not greater than about 1000 mg, not greater than about 900 mg, and not greater than about 800 mg per mammalian subject per day wherein the lower limit and the upper limit are selected independently and in some embodiments, the range of DHA is from about 100 to about 1000 mg, or about 200 to about 900 mg, or about 400 to about 800 DHA mg per mammalian subject per day. By way of specific example, taking into consideration doses that are considered safe for human consumption, a beneficial nutritional supplement in accordance with the present invention might include, for example, 200-500 mg gamma-tocopherol or a gamma-tocopherol enriched composition, once daily; 100-300 mg each quercetin; hesperetin divided between two to three daily doses; and 400-800 DHA divided between two to four daily doses. Such a specific dosing regimen forms part of the invention.

The below are illustrative compositions encompassed within the present invention given as total mgs per day administered to a mammalian subject. In the below examples, the components may be administered together in one composition or administered separately in two or multiple compositions simultaneously or staged throughout the day.

Composition I

300 mg of mixed tocopherols (180 mg gamma-tocopherol; 30 mg alpha-tocopherol; and 90 mg delta-tocopherol); 100 mg hesperetin and 200 mg quercetin.

Composition II

300 mg of mixed tocopherols (180 mg gamma-tocopherol; 30 mg alpha-tocopherol; and 90 mg delta-tocopherol); 100 mg hesperetin; 200 mg quercetin; and 800 mg docosahexaenoate (DHA).

Composition III

300 mg of a gamma-tocopherol enriched composition (greater than 270 mg gamma-tocopherol); 100 mg hesperetin and 200 mg quercetin.

Composition IV

300 mg of a gamma-tocopherol enriched composition (greater than 270 mg gamma-tocopherol); 100 mg hesperetin, 200 mg quercetin, and 800 mg docosahexaenoate (DHA).

Activity of a composition of the present invention, or activity of components administered in methods of the present invention, can be experimentally tested, for example, in an assay which measures the ability of the composition to reduce CRP levels. Assays which measure the ability of a test composition to ameliorate injury(ies) or damage associate with post-exercise muscle injury in vivo are detailed in Examples.

It is understood that the foregoing ranges of components of formulations of the invention may be varied independently (e.g., low tocopherol/high DHA, High tocopherol/low DHA, low tocopherol/low DHA), and that the exemplary combinations described herein should not be construed to limit the invention.

III. Inflammatory Conditions

The present invention is directed to methods and formulations for treating and/or ameliorating inflammation and symptoms of inflammatory conditions. The invention is particularly directed at reducing certain biochemical markers associated with inflammation, many of which have been implicated as adverse prognostic indicators of subsequent complications of such conditions, such as, for example cardiovascular disease. This section will describe exemplary inflammatory conditions that may be improved by administration of formulations of the present invention, as well as biochemical markers that provide indicators of such conditions.

As mentioned above, inflammation is associated with a number of conditions, including cardiovascular diseases or disorders; neurodegenerative diseases such as, Alzheimers; infectious disease, such as, for example, myocarditis, cardiomyopathy, acute endocarditis, pericarditis; atherosclerosis; SIRS/sepsis; ARDS; asthma; rheumatoid arthritis, osteoarthritis, SLE; AHR; bronchial hyperreactivity; COPD, CHF; inflammatory complications of diabetes mellitus; metabolic syndrome; end stage renal disease (ESRD); muscle fatigue or inflammation; and dermal conditions. While the particular symptoms and complications may vary, the conditions discussed in detail below are exemplary of inflammatory conditions that are amenable to treatment by formulations of the present invention. Exemplary treatment paradigms and outcomes are described in below in the Examples.

Muscle Inflammation. Inflammation can be induced by acute exercise in untrained individuals (Jenkins, et al., 1993, Med Sci Sports Exerc 25: 213-7). Inflammation stimulates polymorphonuclear leukocytes and macrophages that produce large amounts of lipid peroxidation products. These peroxidation products are postulated to cause significant damage to DNA and to several other biomolecules in vivo, including, enzymes and lipid membranes (Leeuwenburgh et al. 1999, Free Radic. Biol. Med. 27:186-92; Powers et al., 1999, Med. Sci. Sports Exerc. 31:987-97; Fielding et al., 2000, Med. Sci. Sports Exerc. 32:359-64).

Inflammation can be induced by acute exercise in untrained individuals. By way of example, acute exercise may stimulate polymorphonuclear leukocytes and macrophages, which then produce large amounts of lipid peroxidation products. Without being bound by theory, these peroxidation products are postulated to cause significant damage to DNA and to several biomolecules in vivo, including, enzymes and lipid membranes. In addition, lipid peroxidation products are potentially involved in the actual tissue damage associated post-exercise muscle injury.

Eccentric exercise presents an acute condition characterized by severe inflammation (MacIntyre et al., 2000, Eur. J. Physiol. 81:47-53). An example of an eccentric exercise is weight resistance arm exercises, which have been shown to increase neutrophil migration into the skeletal muscle after such injury (Fielding et al., Supra, MacIntyre et al., Supra). Severe inflammation, pain, and a decrease in range of motion characterize this type of injury. In studies carried out in support of the present invention, an experimental model of exercise-induced inflammation was used to test and demonstrate efficacy of formulations of the present invention in young male volunteers, as detailed in Example 3 herein.

In studies carried out in support of the present invention, subjects who received a formulation of the invention in conjunction with the exercise regimen exhibited reduced levels of IL-6 and CRP, compared to control-treated counterparts.

Diabetes. Diabetes mellitus is a chronic disorder affecting carbohydrate, fat and protein metabolism. Of the two major forms of diabetes, one form, insulin-dependent diabetes (IDDM) (also known as insulin-sensitive diabetes, type I or juvenile diabetes) is caused by under-production of insulin in the pancreas. By far the most prevalent form of diabetes is so-called “adult-onset” or Type II diabetes (non-insulin dependent diabetes, NIDDM). Secondary diabetes is associated with other conditions, including pancreatic disease (e.g. chronic pancreatitis), endocrine diseases (e.g. acromegaly or Cushing's disease), and certain medications or toxins (e.g. thiazides, glucocorticoids). Polycystic ovary syndrome is also associated with elevated insulin levels, insulin resistance or diabetes. Gestational diabetes includes glucose intolerance with the onset of pregnancy, usually at 24-30 weeks gestation (Nathan 1993 Ch. 9(IV) in Scientific American Medicine Rubenstein & Federman, eds., Scientific American, Inc., New York).

All forms of diabetes are characterized by end-organ damage in later phases of the disease, and it is now becoming apparent that inflammation contributes to this aspect of the condition. Without being bound to a particular mechanistic theory, one possible factor is accelerated production of reactive oxygen species known to occur in diabetics, and in particular the increased formation of isoprostanes, which may impair insulin action via reduction of membrane arachidonate and increased inflammatory mediators. These various processes can be assessed by a variety of surrogate markers.

The long term complications of diabetes include numerous vascular conditions, macrovascular, microvascular, and neurologic secondary diabetes and gestational diabetes. While the etiology and ultimate causes of diabetes mellitus vary, the complications linked to the associated metabolic dysfunction and the complications which arise therefrom are common to all types. Common complications include microvascular, neurologic and macrovascular conditions. Complications such as retinopathy and nephropathy are specific for diabetes. Nephropathy associated with diabetes may lead to pre-end stage renal disease (ESRD) and ESRD.

In studies carried out in accordance with the present invention, diabetic patients (type II diabetes) who received formulations of the present invention exhibited reduced levels of HbA1c, in comparison with placebo-treated control subjects.

End-Stage Renal Disease (ESRD). ESRD is associated with known cardiovascular disease risk factors such as hypertension, hyperlipidemia and diabetes mellitus. Cardiovascular mortality rate in dialysis patients is 10 to 20 times higher than the general population, and the 5-year survival rate after initiation of hemodialysis is less than 50%. Recently, attention has been focused on evidence that dialysis patients also have elevated circulating markers of oxidative stress and inflammation, both of which are associated with accelerated atherosclerosis. No therapeutic or preventive agents targeting reduction of these proposed risk factors has proven to reduce mortality in this at-risk population.

Clinical evidence of this effect of oxidative stress inducing increased in HbA1c independent of blood glucose level comes from end-stage renal disease, where increased levels of HbA1c have been observed in non-diabetic patients. Studies carried out in support of the present invention indicate that patients receiving a gamma-tocopherol enriched formulation exhibit a reduction in CRP levels, compared to placebo-treated control subjects. In addition, such subjects may experience increased levels of pre-albumin and albumin. Metabolic Syndrome. Insulin resistance is a common characteristic underlying multiple cardiovascular disease (CVD) risk factors, including hypertension, dyslipidemia, and obesity, as well as type-2 diabetes. Metabolic syndrome can be considered to be an inflammatory condition, and is now defined as a convergence of these predictors—specifically at least three of the following (blood pressure >130/>85 mm Hg; triglyceride >150 mg/dl, HDL-cholesterol <40 and <50 for men and women, respectively; waist >40 and >35 inches for men and women, respectively; fasting glucose >110 mg/dl). These risk factors for metabolic syndrome are highly concordant; in aggregate they enhance risk for CVD.

Cardiovascular Inflammation. Myocarditis and cardiomyopathy are a group of diseases primarily of the myocardium which do not result from hypertensive, congenital, ischemic, or valvular heart disease. These conditions result from an immune response against the myocardium, including lymphocytic infiltration and inflammation. This immune response can occur secondary to infectious diseases such as Chagas' disease (American trypanosomiasis), toxoplasmosis, trichinosis, ricksettal infection (typhus, Rocky Mountain spotted fever), fungal infections, and metazoan parasites; or secondary to autoimmune diseases such as rheumatic fever, rheumatoid arthritis, systemic lupus erythematosus, progressive systemic sclerosis, and polyarteritis nodosa. The immune response leading to myocarditis can be idiopathic in nature as seen in Fiedler's myocarditis. Additionally, myocarditis can be caused by drug reaction to penicillin or sulfonamide, for example. See U.S. Pat. No. 5,496,832. Myocarditis generally defines acute myocardial disease characterized by inflammation, and cardiomyopathy defines more chronic myocardial diseases in which the inflammatory features are not conspicuous. Myocarditis and cardiomyopathy can lead to fever, chest pain, leukocytosis, increased erythrocyte sedimentation rate, left ventricular failure, arrythmias, heart block, ECG changes, and eventually cardiac failure. See U.S. Pat. No. 5,496,832.

Acute pericarditis is defined as an inflammatory disease of the visceral or parietal pericardium and can occur secondary to bacterial, viral (especially echovirus, and Coxsackie Group B), or fungal infection, and can accompany systemic diseases such as rheumatoid arthritis, systemic lupus erythematosus, scleroderma, and uremia. Pericarditis can also occur after cardiac trauma or cardiac surgery that is suggested as being caused by immunologic hypersensitivity. Acute pericarditis can lead to chronic constrictive pericarditis, pericardial tamponade, effusion, and hemorrhage, all of which can result in cardiac failure. See U.S. Pat. No. 5,496,832.

Inflammation, particularly macrophage-mediated and chronic inflammation, has been cited as central to atherosclerosis (U.S. Pat. Nos. 5,877,203 6,210,877) and may serve as a prognostic marker for heightened risk of myocardial infarction (Boisvert et al. 1998 J. Clin. Invest. 101(2):353-363). Atherosclerosis (also known as arteriosclerosis) is the term used to described progressive luminal narrowing and hardening of the arteries. This disease process can occur in any of the arteries in the body leading to a variety of conditions including stroke (hardening or narrowing of arteries leading to the brain), gangrene (hardening or narrowing of peripheral arteries) and CAD (hardening or narrowing of arteries supplying the myocardium). CAD can in turn lead to myocardial ischemia or myocardial infarction. Cardiovascular disorders associated with atherosclerotic disease (and therefore inflammation) can include, for example, myocardial infarction, stroke, angina pectoris and peripheral arteriovascular disease. Macrovascular complications, including atherosclerosis and related conditions are often complications associated with diabetes and metabolic syndrome.

Luminal narrowing of the arteries is the result of the depositions of atheromatous plaque. The plaque consists of a mixture of inflammatory and immune cells, fibrous tissue and fatty material such as low density lipids (LDLs), modifications thereof and α-lipoprotein. The initial causes of atherosclerosis are not completely understood, but it has been suggested that the pathogenesis may include the following stages: endothelial cell dysfunction and/or injury; monocyte recruitment and macrophage formation; lipid deposition and modification; vascular smooth muscle cell proliferation; and synthesis of extracellular matrix. Recent reports have implicated CRP levels with increased risk of cardiovascular disease (Ridker, P. M., et al., N Engl.J. Med. 347(20): 1557-1565).

Respiratory Inflammatory Conditions. Trauma or infection may result in acute life-threatening conditions which include systemic inflammatory response syndrome (SIRS), or adult respiratory distress syndrome (ARDS). When SIRS is caused by infection, it is termed sepsis, which in turn has progressively severe stages (severe sepsis and septic shock). SIRS/sepsis may also result from numerous sources, including bacterial, viral, parasitic, rickettsial or fungal infection, and/or SIRS resulting from non-infectious causes such as burns, pancreatitis, multitrauma, severe surgical trauma, transplant rejection, marked autoimmune rejection, ischemia reperfusion, transfusion reaction or heat stroke. The marked augmentation of pro-inflammatory cytokines which leads to SIRS may also lead to multiple organ dysfunction syndrome (MODS) (e.g. varying degrees of fever, hypoxemia, tachypnea, tachycardia, endothelial inflammation, myocardial insufficiency, hypoperfusion, altered mental status, vascular collapse, which may lead to ARDS, coagulopathy, cardiac failure, renal failure, shock and/or coma).

Chronic asthma can be considered to be predominantly an inflammatory disease with associated bronchospasm. The degree of reactivity and narrowing of the bronchi in response to stimuli is greater in asthmatics than in normal individuals. Persistent inflammation is responsible for the bronchial hyperreactivity or airway hyperresponsiveness (AHR). Mucosal edema, mucus plugging and hypersecretion may also be present and pulmonary parenchyma is normal. Airway narrowing may reverse spontaneously or through treatment. Type 1 (immediate) immune responses may play an important role in the development of asthma in children and many adults; however, when onset of disease occurs in adulthood, allergic factors may be difficult to identify. Exposure to cold dry air, exercise and other aggravating factors may also trigger asthma.

Bronchial hyperreactivity (or airway hyperreactivity, AHR) is a hallmark of asthma and is closely related to underlying airway inflammation. Worsening of asthma and airway inflammation is associated with increase in bronchial hyperreactivity, which can be induced by both antigenic and non-antigenic stimuli. Beta₂-adrenergic agonists are potent agents for the treatment of bronchospasm, but have no effect on airway inflammation or bronchial hyperreactivity. In fact, chronic use of beta₂-adrenergic agents alone, by causing down regulation of beta₂-receptors, may worsen bronchial hyperreactivity. At present, corticosteroids are the one of the most effective agents available which diminish bronchial hyperreactivity. Although inhaled corticosteroids are relatively safe in adult patients with asthma, these agents have tremendous toxicity in children, including adrenal suppression and reduced bone density and growth.

While asthma was once thought of as a disease associated primarily with morbidity, it is now being recognized that asthma is more often associated with mortality than generally thought. In the United States, the annual mortality for asthma, among persons 5 to 34 years is 0.4 per 100,000 people. Deaths are most likely the result of asphyxiation caused by inadequately treated airflow obstruction and generally occur outside of the hospital (Leatherman et al., 1992 Ch. 14(II) in Scientific American Medicine Rubenstein, E. and Federman, D. D. eds. Scientific American, Inc., New York).

Inflammation is also associated with pulmonary or respiratory conditions other than asthma, including adult respiratory distress syndrome (ARDS), an acute and life threatening disease which can lead to multiple organ dysfunction (MOD) (U.S. Pat. No. 5,780,237), and chronic obstructive pulmonary disease (COPD) which is often a complication of cystic fibrosis (Kennedy 2001 Pharmacotherapy 215:593-603). ARDS is a classic example of a restrictive diffuse pulmonary disease while COPD and asthma are exemplary of an obstructive (or airway) disease. Obstructive diseases are characterized by an increase in resistance to air flow due to partial or complete obstruction, while restrictive diseases are characterized by reduced expansion of lung parenchyma and a decreased total lung capacity. COPD (also known as COAD, chronic obstructive airway disease) refers to a group of conditions, emphysema, chronic bronchitis, bronchial asthma and bronchiectasis, which are accompanied by chronic or recurrent obstruction to air flow within the lung (Cotran et al., “Robbins Pathologic Basis of Disease” 4th Ed. 1989, W. B. Saunders Co., Philadelphia, Pa.).

ARDS (also known as acute respiratory distress syndrome) is defined as respiratory failure in adults or children that results from diffuse injury to the endothelium of the lung (as in sepsis, chest trauma, massive blood transfusion, aspiration of the gastric contents, or diffuse pneumonia) and is characterized by pulmonary edema, respiratory distress and hypoxemia (Merriam-Webster's Medical Desk Dictionary 1996 Merriam-Webster, Inc. Springfield, Mass.). ARDS can be due to either trauma or infection and generally occurs in a clinical setting. CF pulmonary disease is characterized as multi-factorial, involving a cycle of airway obstruction, chronic infection and excessive local inflammation that leads to development of bronchiectasis (Kennedy supra), which can be a chronic inflammatory or degenerative condition of the bronchi or bronchioles. Uncontrolled, chronic inflammation directly damages the airway wall, which leads to bronchiectasis and decline in pulmonary function.

IV. Biomarkers Associated with Inflammatory Conditions

Formulations of the present invention are effective in altering one or more biomarkers and/or symptoms of inflammatory conditions. This section will describe exemplary markers which are found to be abnormally high (or, in some cases, low) during acute or chronic inflammatory states. According to an important feature of the present invention, formulations of the present invention, when administered to a subject suffering from an inflammatory condition, will reduce inflammation and/or markers of inflammation, as evidenced by a return toward normal of such inflammatory markers or biomarkers.

A number of proximal mediators of the inflammatory response have been identified and include the inflammatory cytokines, interleukin-1β (IL-1β) (U.S. Pat. No. 6,210,877), IL-1 through IL17, and tumor necrosis factor alpha (TNF-α), as described in U.S. Pat. Nos. 5,993,811 6,210,877 and 6,203,997. Other molecules have been reported for use as markers of systemic inflammation, including for example, CRP (Ridker et al. 2000 N. E. J. M. 342(12):836-43; Spanheimer supra); certain cellular adhesion molecules such as sICAM-1 (U.S. Pat. No. 6,049,147); and B61 (U.S. Pat. No. 5,688,656), e-selectin (also known as ELAM), sICAM, integrins, ICAM-1, ICAM-3, BL-CAM, LFA-2, VCAM-1, NCAM and PECAM. Other proteins associated with inflammation include leukotriene, thromboxane, and isoprostane. Other markers of inflammation include, but are not limited to neopterin; serum procalcitonin; leukotriene, thromboxane, and isoprostane. Other proteins or markers associated with inflammation include serum amyloid A protein, fibrinectin, fibrinogen, leptin, prostaglandin E2, serum procalcitonin, soluble TNF receptor 2, erythrocyte sedimentation rate, and elevated white blood count, including percent and total granulocytes (polymorphonuclear leukocytes) monocytes, lymphocytes and eosinophils.

C-reactive protein (CRP) serves as an exemplary marker for systemic inflammation. See U.S. Pat. No. 6,040,147. In humans CRP levels are elevated during inflammatory disorders such as infection, trauma, surgery, tissue infarction, and in IDDM patients without macrovascular disease. The magnitude of the increase varies from about 50% to as much as 100-fold during systemic inflammation (Gabay, C., et al., New Engl. J. Med. 340: 448-454, 1999). Recent evidence has shown that CRP is also a risk factor for cardiovascular disease and stroke where inflammation plays an important role (Lagrand, W. K., et al, Circulation 100: 96-102, 1999). Most CRP production is from hepatocytes in response to pro-inflammatory cytokines, especially interleukin-6 and 1□ (Ganter, U., et al., EMBO J. 8: 3773-3779, 1989), although macrophages have also been reported to release CRP (Dong, Q, et al, J. Immunol. 156: 481504820, 1996).

In particular, elevated levels of CRP are associated with cardiovascular diseases and disorders, infectious diseases, such as, myocarditis, cardiomyopathy, acute endocarditis, or pericarditis; SIRS; diabetes; metabolic syndrome, as well as other forms of systemic inflammation. Elevated levels of cellular adhesion molecules are associated with systemic inflammation. Elevated levels of IL-1 and TNF-alpha are associated with IDDM and NDDM associated inflammation. Elevated levels of IL-10 and IL-6 are associated with SIRS. Elevated levels of neopterin are associated with SIRS. Elevated levels of procalcitonin are associated with systemic inflammation. Other proteins or markers associated with inflammation include serum amyloid A protein, fibrinectin, fibrinogen, leptin, prostaglandin E2, serum procalcitonin, soluble TNF receptor 2, erythrocyte sedimentation rate, and elevated white blood count, including percent and total granulocytes (polymorphonuclear leukocytes), monocytes, lymphocytes and eosinophils.

It has been reported (Spanheimer, 2001, Postgrad. Med. 109(4) 26) that diabetes may lead to a chronic, low-grade inflammatory state possibly caused by glycosylation of proteins that activate macrophages or by increased oxidative stress. One surrogate marker of inflammation in diabetic patients is glycosylated hemoglobin (HbA1c). Increased levels of HbA1c are associated with end-stage diabetic complications and are predictive of survival in diabetic patients. HbA1c is formed via a multi-step nonenzymatic reaction of glucose and hemoglobin, a process that may be facilitated by oxidative stress. While the mechanism by which reactive oxygen species (ROS) increase and antioxidants decrease HbA1c is unknown, a number of steps in the synthetic pathway may be facilitated by oxidants or slowed by antioxidants.

Pharmaceutical interventions that target blood glucose reduction through various mechanisms consistently reduce HbA1c levels. There is, however, evidence that HbA1c can be lowered without also reducing blood glucose. Further, oxidative stress appears to play an important role in the formation of advanced glycation endproducts (AGEs) that are formed from glycosylated hemoglobin and related compounds. These AGEs remain irreversibly bound to macromolecules and can covalently crosslink to nearby amino groups. The formation of AGEs on long-lived connective tissue accompanies normal aging, and this process occurs at an accelerated rate in diabetics. For example, increased AGEs in collagen are associated with early onset retinopathy and proteinuria.

In this perspective, both HbA1c and AGE's are inflammatory biomarkers of both increased cellular glucose and increased cellular ROS. The reduction of HbA1c without reducing blood glucose may therefore result from a redox active treatment, and implies further benefit in terms of reduced formation of AGEs. Additional biomarkers of inflammation in diabetes include arachidonate (5,8,11,14 eicosatetraenoic acid, an essential omega-6 highly unsaturated fatty acid that provides both critical structural properties to membranes, and which, when released from phospholipids, functions as the primary substrate for eicosanoid (prostaglandin, thromboxane, leukotriene) synthesis. Arachidonate has been linked to many processes that are implicated in type-2 diabetes, such as insulin release from the pancreas, insulin action in skeletal muscle and insulin sensitivity.

V. In vivo Tests and Assays for Inflammatory Biomarkers

This section describes exemplary in vitro and in vivo assays and models that may be used to qualify and/or optimize formulations of the present invention prior to administering such formulations to humans. In general, such assays may provide guidance as to dosing of formulations of the invention.

Assays for the various inflammatory biomarkers are known in the art. For example, reagents for assays for C-reactive protein, may be purchased from CalBiochem (San Diego, Calif.). B61 is an inflammatory marker that is secreted by endothelial cells, fibroblasts and keratinocytes in response to lipopolysaccharide and the pro-inflammatory cytokines IL-1 and TNF. The B61 gene product is highly specific to inflammation (U.S. Pat. No. 5,688,656). The presence of B61 transcript can be detected directly by in situ hybridization using probes of encoding cDNA, according to methods known in the art. Alternatively, the B61 protein can be measured in biological fluids such as plasma, cerebrospinal fluid or urine using an antibody-based assay. These assay procedures known in the art and described in particular in U.S. Pat. No. 5,688,656 are useful in both prognostic and diagnostic applications.

In studies carried out in support of the present invention, a combination of Interleukin-1β, IL-6, and dexamethasone is used to induce CRP production, and counter-agents are tested for their ability to reduce this production in cultured liver cells, as detailed in Example 1A. The assay is performed on cells grown in 96-well format allowing high throughput screening of compounds. As described herein, formulations enriched in gamma-tocopherol, beta-tocopherol and delta-tocopherol reduce CRP levels in an assay such as the one described in Example 1A.

Another useful cell screening assay, exemplified herein in Example 1B, is the E-selectin (ELAM) production assay, which measures activity of test compounds in reducing expression of ELAM in activated endothelial cells. Briefly, endothelial cells are activated by adding known activators such as lipopolysaccharide, TNF, or IL-1β, alone or in some combination. Activated cells produce ELAM, which can be measured using, for example, an E-selectin monoclonal antibody-based ELISA assay. In studies carried out in support of the present invention, ELAM production was decreased by formulations containing enriched forms of gamma-tocopherol, beta-tocopherol, and delta-tocopherol but not by formulations enriched in alpha-tocopherol.

In vivo evaluation of anti-inflammatory activity can be determined by well characterized assays such as reduction of carrageenan-induced paw edema in rats (Gabor, M., Mouse Ear Inflammation Models and their Pharmacological Applications, 2000). Carrageenan-induced paw edema is a model of inflammation, which causes time-dependent edema formation following carrageenan administration into the intraplantar surface of a rat paw. In studies carried out in support of the present invention, gamma-tocopherol-enriched formulations, given orally to rats as a 10-100 mg/kg oral pretreatment over 3 days significantly reduced IL-6 levels in the edematous fluid in this model.

U.S. Pat. No. 6,040,147 describes both prognostic and diagnostic applications of the measurement of levels of particular molecules including certain cytokines (e.g. interleukins 1-17) and cellular adhesion molecules (e.g. sICAM, integrins, ICAM-1, ICAM-3, BL-CAM, LFA-2, VCAM-1, NCAM and PECAM). The presence of such markers may be determined by methods well known in the art, including ELISA (enzyme linked immunosorbent assay) and other immunoassays and can be measured in body fluid, for example, blood, lymph, saliva and urine. U.S. Pat. No. 6,180,643 also describes the use of molecules such as IL-1, TNF-α as markers of IDDM and NDDM in particular, where certain therapies involve inhibiting the production of these molecules

A correlation between SIRS/sepsis and certain tissue or serum markers have also been disclosed, including C-reactive protein (CRP) and neopterin. Serum procalcitonin (ProCT, also termed PAN-116) has recently been described as a clinical marker for systemic inflammation (U.S. Pat. No. 5,993,811) and U.S. patent application Ser. No. 20010007022 describes in detail the use and preparation of antibodies to ProCT (or pCT) in both the therapy and detection of SIRS. Other cytokines which have been suggested as markers for SIRS include interleukin-10 (IL-10) and interleukin-6 (IL-6) (U.S. Pat. Nos. 6,103,702 and 6,203,997).

U.S. Pat. No. 5,496,832, incorporated herein by reference, describes in detail a rat model of immune mediated myocarditis in humans. The model is reproduced in brief below and can be used for testing a non-alpha-tocopherol formulation of the present invention.

Briefly, test rats are immunized with a subcutaneous injection in the footpad of 100 micrograms of porcine cardiac myosin to induce myocarditis, and then treated with test compound. Seven days later, the rats are re-immunized with the same myosin concentration in the contralateral foot pad. Intraperitoneal administration of test compound is initiated on the first day of immunization and is maintained daily for fourteen days. The rats in Group 1 (n=10), received an i.p. injection of vehicle alone daily for fourteen days. The rats in Group 2 (n=7) are not immunized, but received a fourteen day daily i.p. regimen of test compound.

Severity of myocarditis is assessed by analysis of electrocardiograms (ECGs) measured according to standard procedures known in the art.

On the final day, animals are anesthetized with an i.p. injection of pentobarbital, weighed, and final ECGs are obtained. Organs (heart, spleen, right kidney and liver) are removed and macroscopic evaluation of the organs is achieved through application of a standardized gross pathology scoring system. Cardiac sections are made using standard histochemical procedures, and microscopic evaluation of cardiac tissue is carried out to determine effects of treatment paradigms on inflammatory conditions.

U.S. Pat. No. 5,780,237, incorporated herein by reference, describes a diagnostic assay for SIRS, ARDS, sepsis, and MODS based on determining the levels of selected unsaturated and saturated free fatty acids (FFA) in a body fluid and determining a ratio value comprising the sum of the unsaturated FFAs divided by the sum of the saturated FFAs. The unsaturated FFAs include linoleate, oleate, arachinonate and the saturated FFAs include myristate, palmitate, stearate.

Animal Model of SIRS/Sepsis

In vivo animal models of SIRS/sepsis are known in the art and may be used to determine the efficacy of formulations or compositions of the invention, or treatment protocols. As described in detail in U.S. Pat. No. 6,103,702 and briefly described here, one such model in the rat uses a model of chronic peritoneal sepsis that results in systemic inflammatory response syndrome (SIRS). Sepsis is induced under pentobarbital anesthesia in each rat by intraperitoneal (ip) injection of rat cecal contents mixed as a slurry in 5% dextrose in water. Polyethylene catheters (Intramedic PE-50, Baxter, Deerfield, Ill.) are inserted into the right internal jugular vein and right carotid artery. The jugular catheter is used for venous access (drug infusions; volume replacement, etc). The carotid catheter is used to obtain arterial blood samples, and to monitor arterial blood pressure and heart rate. This model of SIRS/Sepsis is associated with elevated concentrations of tumor necrosis factor alpha (TNF-α). The efficacy of treatment in vivo may be determined through monitoring the level of TNF-α in tissues such as spleen and liver or in serum as described in detail in U.S. Pat. No. No. 6,103,702, and briefly described below.

Serum and tissue tumor necrosis factor-alpha (TNF-α) concentrations are determined by enzyme-linked immunosorbant assay (ELISA) according to methods well known in the art. Samples of serum, liver, and spleen are collected, rapidly weighed, and frozen in liquid nitrogen. On the day of assay, tissues are added to labeled tubes containing lysis buffer containing proteases and are immediately homogenized using five 3 sec bursts, washing grinding pistol (3×) between samples with phosphate buffered saline. Samples are then centrifuged for 20 min at 2200 RPM, 4° C. The supernatant is removed and used for TNF-α measurements.

A number of cytokines, such as tumor necrosis factor alpha (TNF-α), interleukin-1β, -6 and/or -8 (IL-1β, IL-6, IL-8) have been implicated in the mediation of inflammation associated with ARDS and asthma (U.S. Pat. No. 6,180,643). Both TNF-α and IL-1 are pro-inflammatory cytokines whose elevated levels over basal have been implicated in mediating or exacerbating both asthma and ARDS as well as other inflammation-associated conditions. Thus, as is known in the art and described in greater detail in U.S. Pat. No. 6,180,643, these molecules may be used as markers for the presence of such conditions as well as in the screening for formulations which ameliorate conditions such as asthma and ARDS. In particular, assays designed to measure the inhibition of the production of TNF-α and IL-1β by test compounds can be used to screen for effective treatments.

Models and protocols for determining the efficacy of treatments for conditions associated with pulmonary or respiratory inflammation are known in the art (e.g. U.S. Pat. Nos. 6,193,957; 6,051,566; 5,080,899, 6,180,643, 6,028,208 and U.S. patent application Ser. Nos. 20010000341, 20010006656). In addition, U.S. Pat. App. Ser. 20010004677 describes a method and apparatus for measuring pulmonary stress. U.S. Pat. No. 6,193,957, incorporated by reference, describes in detail an in vivo model in sheep of pulmonary airflow resistance. The sheep are characterized as dual responders. The model is described in brief below.

Allergic sheep with previously documented dual bronchoconstrictor response to Ascaris suum antigen are used. The sheep are intubated with a cuffed nasotracheal tube and pulmonary airflow resistance (R_{I}) is measured by the esophageal balloon catheter technique, while thoracic gas volume is measured by body plethysmography. Data are expressed as specific R_{L} (SR_{L}, defined as R_{L} times thoracic gas volume (V_{tg})). To assess airway responsiveness, cumulative dose-response curves to inhaled cabachol are constructed. Airway responsiveness is measured by determining the cumulative provocation dose of carbachol, according to standard methods detailed in the reference. Each animal's baseline airway responsiveness is determined, and then on different experimental days the sheep undergo airway challenge with Ascaris suum antigen. Active anti-inflammatory test agents will reduce pulmonary stress in this model.

By way of further example, U.S. Pat. No. 6,051,566, incorporated herein by reference, describes in detail protocols for studies of non-specific bronchial hyperreactivity in patients. U.S. Pat. No. 5,080,899 details a in vivo guinea pig model for studying the efficacy of orally administered drugs for the treatment of pulmonary inflammation. The model is described in brief below.

Male Hartley guinea pigs are sensitized with an intramuscular (i.m.) injection in each hind leg of ovalbumin. Following a 3 week sensitization period, each animal is pretreated with pyrilamine to prevent hypoxic collapse and death, and then challenged with an aerosol of 0.2% OA for 3 min using a DeVilbiss Ultra-Neb 100 nebulizer. Test formulations or vehicle are administered orally in a volume of 1 ml/500 g body wt. at appropriate times pre- and post-challenge. A test formulation is administered orally at—various times prior to aerosol treatment. After sacrifice, the trachea of each animal is isolated and the lungs are lavaged with isotonic sterile saline. This bronchoalveolar lavage fluid from each animal is examined for inflammatory cells present therein, using a Coulter model ZM particle counter (Beckman Coulter, Inc., Fullerton, Calif.).

The effect of test compounds in the treatment of chronic obstructive pulmonary disease can be tested in a murine model of pulmonary neutrophilia induced by lipopolysaccharide via intranasal instillation. Bacterial lipopolysaccharide (LPS) is a macromolecular cell surface antigen of bacteria which, when applied in vivo triggers a network of inflammatory responses. The main characteristics of this LPS-induced lung inflammation model, macrophage activation, tumor necrosis factor-alpha (TNF-α) production and neutrophil infiltration and activation, are features of chronic obstructive pulmonary disease. This model causes pulmonary inflammation as an acute injury which occurs after 2 to 4 hours in the airway lumen, where all the inflammatory parameters can be assessed by bronchoalveolar lavage (BAL).

As described in U.S. Pat. App. No. 20010000341, a test compound given intranasally to Female Balb/C mice (20-25 g) under anaesthesia. Three hours after intranasal administration of LPS. Subsequently, bronchoalveolar lavage is performed and the fluid is examined for presence of cells. BAL myeloperoxidase (MPO) activity is measured on fresh BAL supernatant using a 96 well plate format colorometric assay, according to standard methods known in the art.

The inhibitory effect of the compound under test on lung inflammation is shown by the reduced neutrophil count and/or reduced MPO activity obtained after administration of the compound compared with that obtained after administration of diluent alone. In a related technique described in U.S. Pat. No. 6,028,208, incorporated herein by reference, a male golden hamster is placed in an inhalation chamber and allowed to inhale LPS generated by an ultrasonic nebulizer for 30 min to cause airway inflammation. Just after the inhalation of the LPS, a test compound is administered through intrarespiratory tract administration or orally under halothane anesthesia. After 24 hr, tracheal branches and pulmonary alveoli are washed, and the number of neutrophils in the washing are determined. Using the number of neutrophils obtained in the absence of a test compound as the control, the decreasing rates of the numbers of neutrophils are expressed in terms of percent suppression based on the control.

This model is widely used as an inflammatory pulmonary disease model (Esbenshade et al., 1982 J. Appl. Physiol. 53:967-976), and it has been reported that the model exhibits a morbid state of acute aggravation of an inflammatory pulmonary disease (Hurlar et al., 1983 J. Appl. Physiol. 54:1463-1468).

U.S. Pat. No. 6,180,643 describes in detail several assays which are used to characterize the ability of compounds to inhibit the production of TNF-α and IL-1□.

Test formulations can also be tested for anti-inflammatory properties in models of inflammation including the carageenan paw edema model (Winter et al 1962 Proc. Soc. Exp. Biol. Med. 111:544; Swingle, in R. A. Scherrer and M. W. Whitehouse, Eds., 1974 Antiinflammatory Agents, Chemistry and Pharmacology, Vol. 13-II:33, Academic, New York) and collagen induced arthritis (Trentham et al 1977 J. Exp. Med. 146:857; Courtenay 1980 Nature (New Biol.) 283:666).

Anti-islet cell antibodies (ICAs) have been suggested as markers of IDDM, being present up to 10 years prior to the clinical manifestation of the disease (Nathan, supra). U.S. Pat. No. 6,057,097 also describes in detail methods for using anti-nuclear auto-antibodies (ANAs) associated with IDDM for prognostic and diagnostic applications.

A TH2-specific gene which encodes a protein (STIF) differentially expressed within the TH2 cell sub-population has been reported as linked to proliferative and T-lymphocyte-related disorders such as chronic inflammatory diseases and disorders including IDDM (U.S. Pat. No. 6,190,909).

U.S. Pat. No. 5,789,652 is directed to a non-insulin dependent diabetic rat which can be used to determine the efficacy of test compounds in the treatment of NIDDM. U.S. Pat. No. 5,877,203 describes in detail the use of cholesterol fed rabbits for modeling the efficacy of a test compound on the binding of monocytes to the thoracic aorta. U.S. Pat. No. 6,261,606 describes several animal models of diabetes, (IDDM, NIDDM and steroid-induced) for use in screening the efficacy of test formulations in the treatment of these conditions. Description of these models is reproduced below in brief.

Streptozotocin Rats—Model for IDDM. (U.S. Pat. No. 6,261,606)

Sprague Dawley male rats weighing 120-130 g are injected subcutaneously with a single dose of streptozotocin (60 mg/kg body weight) in 0.5 ml citrate buffer, 0.05 M pH 4.5. Plasma glucose concentrations are measured seven days later using a commercial glucometer. Animals with blood glucose higher than 250 mg/dl are chosen for the subsequent tests with test compounds. Test compounds are introduced orally. Blood is collected from the tail vein at intervals of 30 min, and levels of glucose, free fatty acids and triglycerides are measured as known in the art. Mirsky 1993 J. Inorg. Biochem. 49:123-128.

Sand Rats and Spiny Mice—Models for NIDDM. (U.S. Pat. No. 6,261,606)

Sand rats (Psammomys obesus) and Spiny mice (Acomys rusatus), when fed a high energy diet, develop NIDDM. Schmidt-Nielsen et al., 1964 Science 143: 689-690. Such models can be used to test non-alpha-tocopherol compositions of the present invention for their ability to reduce symptoms of inflammation associated with NIDDM, including a reduction in the levels of one or more inflammatory markers, such as for example, CRP.

Formulations of the invention may be tested for efficacy in various cellular models of inflammation that are known in the art. For example, E-selectin (also called Endothelial Leukocyte Adhesion Molecule, or ELAM) is a cell adhesion molecule that is actively expressed on the surface of endothelial cells, where it helps mediate the initial attachment of circulating leukocytes. It therefore serves as a sensitive and specific marker of inflammation. Cell assays have been devised to measure the ability of test compounds to reduce expression of E-selectin by endothelial cells that are subjected to inflammatory stimuli, such as lipopolysaccharides and interleukin-1β (IL-1B). Test compounds that inhibit this response have anti-inflammatory properties. Such an assay is described in Example 1B herein; other assays protocols are known in the art. (See, e.g. Hess, D. C., et al. Neursci. Lett. 213(1): 37-40, 1996). Compositions of the present invention can be tested in such an assay for their ability to reduce expression of E-selectin.

VI. Clinical Human Anti-Inflammatory Activity

This section describes exemplary outcomes of formulations of the present invention in human subjects.

Muscle Performance. Example 3 details studies in which a non-alpha-tocopherol enriched formulation prepared in accordance with the present invention was further tested in a model of muscle performance. Briefly, human subjects who are not customarily involved in weight training were given either placebo or a pre-determined daily dose of a non-alpha-tocopherol enriched formulation of the invention, as detailed in the Example. Blood metabolites and inflammatory markers were measured according to well known methods prior to and at defined time intervals after eccentric exercise (for example, a defined arm “curl”) on an exercise machine. Subjective pain assessment was also elicited. Anti-inflammatory tocopherol formulations provided reduction in at least one or more markers of inflammation, as defined herein, as compared to placebo-treated control subjects.

Results of the study demonstrated that 3 days after the exercise challenge, there were increases in CRP, IL-6 and white blood cell count. By 7 days post-exercise, the levels of the inflammatory markers had returned toward baseline levels. The degree of these elevations post-exercise differed according to treatment group, and there was evidence that treatment with formulations of the present invention modulated these effects. That is, after one week of supplementation prior to the exercise, there was a significant reduction of levels of CRP in the treatment group, as compared to the placebo group. Subsequently, following the exercise injury, the treatment group continued to show reduced levels of CRP compared to baseline, whereas the placebo group showed increases in levels of CRP. At 7-days post-exercise, levels of CRP in the placebo group returned to baseline, whereas the levels in the treatment group remained below baseline values. Similarly, there was a significant increase in IL-6 levels 3 days after exercise in the placebo group that was not observed in the treatment group. In addition, in the present study, correlations between baseline levels of CRP and IL-6 were observed. It is noted that IL-6 release by monocytes is the signal for liver CRP production. IL-6 is a cytokine that modulates CRP and recruits macrophages and monocytes. Macrophages are known to secrete IL-6 and a correlation between post-exercise change in white blood cell count and change in IL-6 levels was noted.

The present invention demonstrates that eccentric exercise resulted in acute muscle injury as evidenced by significant increases in markers of tissue damage, namely CK and LDH, as well as by reported increases in pain and decreases in range of motion. These changes in markers of tissue damage occurred in both the treatment group and placebo group. The present invention demonstrates that the administration of a formulation of the present invention results in anti-inflammatory properties and no short term side effects.

Diabetes. Formulations of the invention were tested for their effect on diabetic control and on the levels of isoprostanes and C-reactive protein in individuals with poorly controlled Type II diabetes. Diabetic control was determined by measuring levels of glucose, insulin, and hemoglobin A1c (HbA1c) in the subjects.

Patients were assigned to receive either Patients were assigned to receive a formulation or placebo, as detailed in Example 5, for a total of 8 weeks following a two-week single blind placebo run-in period. The study was powered to assess each formulation against placebo. Patients took a total of 8 capsules per day (6 capsules in the morning, one in the afternoon and one in the evening), and daily glucose levels were recorded. Demographic characteristics, medical history (e.g., duration of DM diagnosis), HbA1C, and urine protein (mg/24 hr) were compared among treatment groups for comparability at randomization into the double-blind phase. Formulations of the invention were tested for their effect on diabetic control and on the levels of isoprostanes and C-reactive protein in individuals with poorly controlled Type II diabetes. Diabetic control was determined by measuring levels of glucose, insulin, and hemoglobin A1c (HbA1c) in the subjects. Increased HbA1c levels are associated with end-stage diabetic complications. The primary foci of this study were oxidative stress and inflammation endpoints. Patients treated with formulations of the present invention (gamma-tocopherol-enriched mixed tocopherols+flavonoids) with or without DHA, exhibited a significant reduction in levels of HbA1c.

End-stage Renal Disease (ESRD). Example 4 provides details of a clinical study in which patients undergoing renal dialysis were given formulations of the present invention and assessed for various symptoms and markers of inflammation. Subjects were randomly assigned into groups to receive either 300 mg γ-tocopherol in gel caps and 800 mg DHA in separate gel caps or an equivalent number of placebo gel caps to be taken daily. Blood was drawn from patients to test: chem panel, CBC, C-reactive protein (CRP), oxidized albumin, protein carbonyls, glycosylated hemoglobin (HbA1c), Interleukin-6 (IL-6), pre-albumin, flow cytometry (IL-1, IL-6, IL-8, TNF-α), γ-tocopherol, 2,7,8-trimethyl-2-(2′-carboxyethyl)-6-hydroxychroman (γ-CEHC), RBC docosahexaenoic acid (DHA) and arachidonate levels, f2-isoprostanes, pre-albumin and albumin at each visit.

Cardiovascular Disease. Patients are screened for CRP and cholesterol levels, as described by U.S. Pat. No. 6,040,147 (Ridker, et al.), incorporated herein by reference, and are selected for inclusion in a prospective trial of formulations of the invention, if their CRP levels and/or cholesterol levels are above a pre-determined value, as discussed in the Ridker patent mentioned above. In a double-blind, randomly assigned trial, blood levels of CRP, cholesterol, and other inflammatory markers are periodically assessed, and incidence of heart attacks (myocardial infarct), stroke (cerebral infarct, cerebral ischemia) is assessed for a period of several months to several years (Ridker, P. M., et al., N Engl. J. Med. 347(20): 1557-1565). Formulations of the present invention are considered effective if CRP and/or cholesterol levels are reduced in this population.

Therefore, the present invention is related to compositions and methods for the treatment and/or amelioration of symptoms of a number of inflammatory conditions, including, but not limited to muscle injury, muscle fatigue, diabetes, metabolic syndrome, ESRD and their respective associated systemic inflammatory response. Without being bound by theory, the compositions of the present invention, or all components of a composition may decrease the potential for development of and/or decrease the presence of amounts of reactive oxygen species as mediators of inflammation.

The above-described compositions and methods of administration are meant to describe but not limit the methods and compositions of the present invention. The methods of producing various compositions and devices are within the ability of one skilled in the art and are not described in detail here.

The formulations of the present invention, and methods using the compositions are capable of inflammatory biomarkers and other symptoms of systemic and acute inflammation, as demonstrated herein.

Various assays, compositions and methods useful for identifying compositions and methods for reducing tissue damage are provided in the Examples. Specific formulations are provided to guide the practitioner in selecting optimal doses, but should not be construed to limit the scope of the invention.

The following examples are provided to illustrate, but not limit, the invention.

### EXAMPLES

### Example 1

Cellular Inflammation Assays

This example provides exemplary assays for measuring inflammatory reaction in a cell line. Specifically, this assay provides a predictive measure of anti-inflammatory activity of compositions of the present invention.

A. Human Hep3B Cells—CRP Assay

Hep3B Cell Line was obtained from the American Type Culture Collection (ATCC Catalog No. HB-8064). The Hep3B cell line was derived from liver tissue of an 8-year-old Black male. The cells are epithelial in morphology and produce tumors in nude mice. The cells produce α-fetoprotein, hepatitis B surface antigen, albumin, α-2-macroglobulin, α-1-antitrypsin, transferrin, plasminogen, complement C3 and β-lipoprotein (Knowles B B, et al., Science, 1980, 209:497-499). This cell line has been widely used to study hepatocyte cytokine and acute phase protein release (e.g., Damtew B, et al., 1993, J Immunol 150:4001-4007).

HEP3B cells are grown in Minimum Essential Medium (MEM; GIBCO) supplemented with 10% Fetal Bovine Serum (FBS; Hyclone), 1× Penicillin/Streptomycin (GIBCO, Cat#. 15140-122) and 0.1 mM non-essential amino acids (GIBCO, Catalog No. 11140-050). Cells are thawed and transferred to warm medium according to standard methods known in the art.

Cells are incubated in flasks at 37° C. with 5% CO₂ in an air atmosphere incubator. HEP3B growth media is changed every 2 days until the cells reach 70-80% confluence (approx. 3-4 days). For assay, the cells are transferred to 96-well plates, seeded at 5000 cells per well in culture media, and left to grow for 7 days in a 37° C. incubator (air supplemented with 5% CO₂). Media is replaced daily until assay.

Test compounds are diluted into “Stimulus Buffer” (MEM medium containing 0.1 mM non-essential amino acids, 1×penicillin/streptomycin, 10% FBS with 10 ng/ml IL-1β, 20 ng/ml IL-6 and 1 μM dexamethasone. Media is removed from the cells and is replaced with 200 μl of test dilution. Cells are returned to the incubator for three days at 37° C. CRP ELISA is then performed on supernatant from the cells, as described below.

Costar EIA/RIA plates are coated with rabbit anti-human CRP (DAKO) diluted 1:4000 in carbonate buffer (100 □l/well) for 45 minutes at 37° C. Plates are then washed 5× with CRP washing buffer (50 mM Tris-HCl, 0.3M NaCl, 0.5 MI Tween-20, pH 8.0) using an automatic plate washer. Plates may be dried, covered and refrigerated until use. Supernatant (100 μl) is removed from each well of the test plates and added to the corresponding well of a pre-coated ELISA plate.

100 μl HRP-conjugated rabbit anti-human CRP (DAKO) diluted 1:500 (in CRP wash buffer) is added to each well, followed by incubation for 30 minutes at 37° C. Plates are washed 5× with CRP washing buffer using the automatic plate washer. 200 μl of 3,3′,5,5′-Tetramethyl Benzidine (TMB) liquid Substrate System (Sigma, St. Louis, Mo.) is added to each well, followed by incubation in the dark for 15 minutes at room temperature. Finally, 50 μl of 1M H₂SO₄ is added to each well and absorbance at 450 nm is immediately measured in a microtiter spectrophotometer.

CRP measured as above is normalized to cell count per well, using a cell viability assay, such as the Cell Tracker Green assay. To do this, the remainder of the medium is from the cell test plates, cells are washed with 200 μl of pre-warmed 1× Hanks Basic Salt Solution (HBSS; GIBCO), and 100 μL of 5 μM Cell Tracker Green (Molecular Probes, Eugene, Oreg.) is added to each well. Plates are then incubated at 37° C. for 30 minutes. Cells are then washed twice with prewarmed 1× HBSS. Plates are immediately read using a Fluoroskan® flourometer with a 485 excitation/538 emission filter pair.

B. Cell-ELAM Assay

Endothelial-Leukocyte Adhesion Molecule (ELAM), also known as E-selectin, is expressed on the surface of endothelial cells. In this assay, lipopolysaccharide (LPS) and IL-1β are used to stimulate the expression of ELAM; test agents are tested for their abilities to reduce this expression, in accordance with studies showing that reduction of leukocyte adhesion to endothelial cell surface is associated with decreased cellular damage (e.g., Takada, M., Et al., Transplantation 64: 1520-25, 1997; Steinberg, J. B., et al., J. Heart Lung Trans. 13:306-313, 1994).

Endothelial cells may be selected from any of a number of sources and cultured according to methods known in the art; including, for example, coronary artery endothelial cells, human brain microvascular endothelial cells (HBMEC; Hess, D. C., et al., Neurosci. Lett. 213(1): 37-40, 1996), or lung endothelial cells. Cells are conveniently cultured in 96-well plates. Cells are stimulated by adding a solution to each well containing 10 μg/ml LPS and 100 pg/ml IL-1β for 6 hours in the presence of test agent (specific concentrations and time may be adjusted depending on the cell type). Treatment buffer is removed and replaced with pre-warmed Fixing Solution® (100 μl/well) for 25 minutes at room temperature. Cells are then washed 3×, then incubated with Blocking Buffer (PBS+2% FBS) for 25 minutes at room temperature. Blocking Buffer containing Monoclonal E-Selectin Antibody (1:750, Sigma Catalog #S-9555) is added to each well. Plates are sealed and stored at 4° overnight. Plates are washed 4× with 160 μL Blocking Buffer per well. Second Antibody-HRP diluted 1:5000 in Blocking Buffer is then added (100 μL/well), and plates are incubated at room temperature (protected from light) for two hours. Plates are then washed 4× with Blocking Buffer before addition of 100 μL of ABTS Substrate solution at room temperature (Zymed, Catalog #00-2024). Wells are allowed to develop for 35 minutes, before measurement at 402 nm in a Fluoroskan® Reader with shake program for 10 seconds. Positive results are recorded as a decrease in ELAM concentration in tested wells, as compared to control wells.

### Example 2

In Vivo Model of Cellular Inflammation

This assay measures the ability of test compounds to prevent or reduce inflammation secondary to oxazolone or arachidonic acid.

A. Arachidonic Acid

Albino male CD-1 mice, 7-9 weeks old were used in this test. A 20% (w/v) arachidonic acid solution in acetone is prepared. Twenty microliters of the arachidonic acid solution is applied to the dorsal left ear of the mouse. Immediately thereafter, test compounds (20 μL in 70% ethanol/30% propylene glycol) are applied to the left ear. The untreated right ears served as control. Mice are sacrificed by CO₂ inhalation, one hour after treatment. The left and right ears are removed and 7 mm punch biopsies taken from each. The punch biopsies are weighed, and the differences calculated.

B. Oxazolone

CD-1 mice are induced by applying 3% oxazolone (Sigma) (30 mg/ml prepared in corn oil:acetone) to the shaved abdomen. Five days later, the mice are challenged with 2% oxazolone (20 mg/ml) in acetone on the left ear (right ear was untreated control). One hour after challenge, test compounds are applied to the left ear in 70% ethanol/30% propylene glycol. Animals are sacrificed 24 hours later and 7 mm ear punches are removed. The ear punches are placed on a balance scale, and the difference between the untreated and treated ears is determined. Percent inhibition is calculated by comparing the means of each group to the vehicle group. (Hydrocortisone serves as a positive control in this test.)

### Example 3—Post-Exercise Muscle Injury

Example 3 provides materials and methods for a study designed to measure the effects of methods of treatment that comprise administration of a gamma-tocopherol enriched composition, hesperetin, quercetin, and DHA on symptoms associated with post exercise muscle injury.

A. Mammalian Subjects

Healthy, non-smoking, young male subjects were recruited for the study to measure the effects of post exercise muscle injury. The subjects could not be involved in a regular weight-training program or have a prior history of injury to the biceps brachii or elbow region. In addition, they were required to be free of vitamin/mineral supplementation for six weeks prior to the study. Forty subjects were randomized and completed the study.

The study involved one study center using a randomized, double blind parallel design involving a test formulation comprising 300 mg of mixed tocopherols (180 mg gamma-tocopherol; 30 mg alpha-tocopherol; and 90 mg delta-tocopherol); 100 mg hesperetin, 200 mg quercetin and 800 mg DHA versus placebo. The subjects (ages 18-35 yr.) were randomly assigned to receive either test formulation (N=20) or placebo (N=20) for 7 days before and then 7 days after an acute bout of eccentric exercise. Subjects reported to the clinic in a fasting condition (minimum of 10 hours). Blood samples were collected on day 0 (start of supplementation), day 7 (exercise), day 10, and day 14 to assess markers of tissue injury (CK and LDH), peroxidation products (isoprostanes), and inflammatory mediators (CRP, IL-6, and WBC). Serum γ-tocopherol levels were used as an objective marker of compliance to taking the formulation. The subjects also completed a subjective evaluation of muscle soreness. A medical history and brief physical examinations (blood pressure, pulse rates and body weight) were conducted for suspected adverse events.

The subjects were instructed to perform three sets of ten repetitions using 80% of their eccentric 1 repetition maximum on the Cybex® arm curl machine using only the non-dominant arm. The subjects were given two minutes rest periods between sets and repetitions continued until fatigue. This type of exercise-induced injury causes severe pain and edema for several days post-injury. Subjects were instructed to maintain current exercise level and not to initiate a weight loss program for the duration of the study.

The ingredients of the composition were administered in multiple formulations: formulations consisted of: 500 mg hard-shell capsules containing 300 mg flavonoids (100 mg hesperetin and 200 mg quercetin) and/or rice powder, dose divided into three capsules daily with meals; 500 mg softgel caps containing 300 mg mixed tocopherols (60% or 180 mg γ-tocopherol, 10% or 30 mg α-tocopherol, and 30% or 90 mg δ-tocopherol), one capsule taken once daily with breakfast, and 500 mg softgel caps containing 200 mg docosahexaenoate (DHA) and/or high oleic sunflower oil, four capsules taken once daily with breakfast (total DHA dose was 800 mg/day). The placebo formulation consisted of high oleic sunflower oil softgel caps and hard-shell rice powder capsules. Blood samples were subjected to the following laboratory analyses: blood chemistries, CK, LDH, isoprostanes, CRP, IL-6, and white blood cell count (WBC). The isoprostanes were assessed using an ELISA method. Serum γ-tocopherol and γ-CEHC metabolite concentrations were analyzed by HPLC.

Demographic characteristics and outcome parameters were compared among treatment groups for comparability at randomization into the double-blind phase. Normality and variance assumptions were examined by an F-test for impact on statistical techniques. If significant deviation from these assumptions was observed, non-parametric methods were employed. Otherwise, continuous variables were analyzed with a non-paired t-test. For outcome efficacy analyses, continuous variables were analyzed by repeated measures analysis of variance (ANOVA) including time and formulation effects. Comparisons between groups for changes in non-normally distributed variables (CRP) were analyzed by Mann-Whitney U Test. Correlations of the biomarkers of inflammation were assessed using Spearman's Rho, a test robust to outliers.

Serum γ-tocopherol metabolite (γ-CEHC) levels (ng/mL) were used as a measure of compliance to taking the test article.

Markers used to assess cellular injury showed an increase after the exercise. Three days post-exercise (T3) versus baseline (T1) CK and LDH showed significant (p<0.0001) increases with no significant differences between groups for CK (p=0.86) or LDH (p=0.57). Levels of CK and LDH returned to baseline levels 7 days after the exercise.

Subjective measures of muscle soreness, pain and range of motion (ROM) were also assessed before and after the exercise. Pain was measured using a visual analogue scale (0 to 10, with 0=no pain and 10=extreme pain) and ROM was measured as active arm flexion using standard goniometry. Pain was significantly (p<0.0001) increased 3 days after the injury in both treatment and placebo groups and was reduced to baseline levels by day 7 post-exercise. ROM was significantly (p<0.0001) decreased 3 days after the injury in both groups and also returned to baseline values by day 7.

At 1 week after supplementation, the treatment group had decreased CRP values vs. placebo (mean±SE, −0.36±0.19 vs. 0.17±0.28, p=0.08) (medians, −0.10 vs. −0.02). Three days after exercise, as compared to baseline (T3-T1), CRP levels decreased in the treatment group (−0.10±0.33) and increased in the placebo group (0.50±0.21) (group differences p<0.01) (medians, −0.00 vs. 0.39). The group differences in CRP levels seven days after exercise as compared to baseline (T4-T1) did not reach significance (−0.27±0.31 vs. 0.16±0.22, p=0.14) (medians, −0.01 vs. 0.06).

Additional markers of inflammation, WBC count and IL-6 levels, were measured pre- and post-exercise. Elevations in WBC count three days post-exercise returned to baseline levels. Three days after the exercise (T3-T2), the treatment group had a different IL-6 response (means±SE) than the placebo group (1.86±3.97 vs. 23.54±12.40, p=0.05) (medians, 0.00 vs. 7.06).

Correlations between the biomarkers of inflammation showed baseline concordance of CRP and IL-6 (p=0.07). In addition changes in IL-6 after exercise (T3-T1) correlated with changes in white blood cell count (p=0.05).

### Example 4—End-Stage Renal Disease (ESRD)

Formulations of the invention were tested for effects on markers of oxidative stress and inflammation in sixty male and female adults with end stage renal disease (ESRD) on chronic hemodialysis. An intervention trial was conducted using a using a randomized, double blind parallel group design involving a the formulation versus placebo over the course of eight weeks, followed one week later by a post-intervention follow-up visit.

The primary study objectives were to assess the effect of the formulation: on the systemic inflammatory biomarker CRP, on the ratio of the EPO dose prescribed to the measured hematocrit level, and on surrogate markers of oxidative stress in adults with end-stage renal disease undergoing chronic hemodialysis (oxidized plasma proteins and carbonyls, and blood HbA1c).

Secondary study objectives were to assess the effect of NIS on pre-albumin and albumin, plasma proteins which are reduced during systemic inflammation, and on serum levels of f2-isoprostanes, a surrogate marker of oxidative stress.

Study Variables included serum C-reactive protein, oxidized plasma proteins, protein carbonyls and whole blood glycosylated hemoglobin (HbA1c). Secondary study variables include albumin, pre-albumin and γ-CEHC in serum, and membrane docosahexaenoic acid and arachidonic acid from monocytes and red blood cells.

Adults age 18-70 with ESRD were included in the study if they were under treatment with chronic hemodialysis and had clinically acceptable hepatic function (transaminases<2times normal), and a white blood cell (WBC) count between 4.5-10.5 K.

Subjects were randomly assigned into one of the following two treatment arms. The first group received two 500 mg γ-tocopherol gel caps (containing 150 mg γ-tocopherol plus high oleic sunflower oil) and four 500 mg DHA gel caps (containing 200 mg of DHA, Martek 40% DHA Dhasco oil) to be taken in the morning, and the second received six 500 mg placebo gel caps (500 mg high oleic sunflower oil) to be taken in the morning. Subjects were instructed to take all prescribed medications as instructed by their physician for the duration of the study. OTC medications and nutritional supplements (within the limits of exclusion) were allowed per the subject's usual practice.

Vital signs and a review of concomitant medications, compliance and adverse events were checked at each visit. Blood draws were done to test: chem panel, CBC, C-reactive protein (CRP), oxidized albumin, protein carbonyls, glycosylated hemoglobin (HbA1c), Interleukin-6 (IL-6), pre-albumin, flow cytometry (IL-1, IL-6, IL-8, TNF-α), γ-tocopherol, 2,7,8-trimethyl-2-(2′-carboxyethyl)-6-hydroxychroman (γ-CEHC), RBC docosahexaenoic acid (DHA) and arachidonate levels, f2-isoprostanes, pre-albumin and albumin at each visit, with the exceptions of the initial screening visit, where only chem panel, CBC, CRP, oxidized albumin, & protein carbonyls were measured, and the post-intervention follow-up visit, where no blood was drawn and only vital signs recorded.

All visits were calculated from the baseline visit. Visits were allowed to be scheduled plus or minus one day of the targeted visit date. A signed informed consent was obtained at the first visit prior to any procedures. Medical history, vital signs, review of concomitant medications, review of inclusion/exclusion criteria, 12-lead resting ECG and blood draw for chem panel, CBC, CRP, oxidized albumin, protein carbonyls were taken at the initial baseline visit.

Methods of Analysis

Baseline Comparability

Demographic characteristics, medical history (eg duration of ESRD diagnosis, percent with diabetic diagnosis), CRP, HbA1c, EPO dose/Hct, oxidized albumin, protein carbonyls. For each parameter, baseline will refer to Visit 2 values, except for CRP, for which baseline will be an average of the values obtained at visits 1 and 2.

Continuous variables are analyzed with an analysis of variance model (ANOVA), including time and formulation effects. Categorical variables are analyzed using Fisher's exact test.

### Example 5 Type II Diabetes

Formulations of the invention were tested for their effect on diabetic control and on the levels of isoprostanes and C-reactive protein in individuals with poorly controlled Type II diabetes. Diabetic control was determined by measuring levels of glucose, insulin, and hemoglobin A1c (HbA1c) in the subjects. Increased HbA1c levels are associated with end-stage diabetic complications. The primary foci of this study were oxidative stress and inflammation endpoints.

Utilizing a randomized, double blind, parallel group design, patients were assigned to receive either: A) 300 mg α-Tocopherol+100 mg Flavonoids (hesperitin & quercetin), B) 300 mg Mixed Tocopherol (60% γ-Tocopherol)+100 mg Flavonoids (hesperitin & quercetin), C) 300 mg Mixed Tocopherol (60% γ-Tocopherol)+100 mg Flavonoids (hesperitin & quercetin)+800 mg DHA+300 mg R/S-Alpha Lipoate, or D) placebo (High Oleic Sunflower Oil capsules+Rice Powder capsules) for a total of 8 weeks following a two-week single blind placebo run-in period. The study was powered to assess each formulation against placebo. Patients took 6 capsules in the morning, one in the afternoon and one in the evening each day, and daily glucose levels were recorded. Demographic characteristics, medical history (e.g., duration of DM diagnosis), HbA1C, and urine protein (mg/24 hr) were compared among treatment groups for comparability at randomization into the double-blind phase. For each parameter, baseline referred to the last measurement prior to study treatment.

Eligibility criteria required patients to be between the ages of 30-55; to have poorly controlled Type II diabetes as defined by a fasting glucose of >140 mg/dl and glycosylated hemoglobin (HBA1C)>7.5%; clinically acceptable hepatic (transaminases<2 times normal) and renal function (creatinine<1.5 mg/dL); WBC between 4.5-10.5 K).

Clinical parameters were assessed at Randomization following the two-week run-in period, then again on Day 7, Day 28 and Day 56. Blood chemistries, lipid panel, CBCs with differential and vital signs (weight, blood pressure and heart rate) and parameters related to diabetic control (fasting glucose, insulin levels and HbA1C) were determined at every visit. A surrogate marker of lipid peroxidation (24 hour urinary isoprostane levels) was assessed at Randomization, Day 28 and Day 56. The 24-hour urine samples were also analyzed for urinary protein and creatinine. Inflammatory markers (CRP, and WBC) were obtained at Randomization, Day 7, Day 28 and Day 56. Samples were collected for gene chip analysis (Randomization, Day 7 and Day 56), compliance parameters (tocopherol and DHA levels) at Randomization and Day 56 and RBCs for fatty acid analysis (Randomization and Day 56).

Blood hemoglobin A1c was determined 4 times during the study, but the primary evaluation for effect was at visit 3 (randomization) and visit 6 (after 8 weeks of intervention). Decreases in HbA1C (compared to placebo) were observed in subjects who received mixed tocopherol+DHA (groups B and C), whereas no changes were observed in the patients who received α-tocopherol (group A). When expressed as percentage of mean change from baseline, the mixed tocopherol groups experienced reductions of 4% and 4% for groups B and C, respectively; whereas the placebo group's values increased by 3%. Combining the values from both mixed tocopherol groups compared to the placebo group and utilizing a one-tail test, the reduction in HbA1c was significant at P=0.05.

It is claimed:

1. A method of reducing the level of an inflammatory biomarker in an individual subject to an inflammatory condition, comprising administering to the individual an effective amount of a formulation comprising a non-alpha-tocopherol and an omega-3 fatty acid.

2. The method of claim 1, wherein the biomarker is selected from the group consisting of C-reactive protein (CRP), interleukin-1-alpha (IL-1-alpha), interleukin-1-beta (IL-1-beta), interleukin-6 (IL-6), and elevated white blood cell count (WBC).

3. The method of claim 2, wherein the biomarker is IL-6

4. The method of claim 2, wherein the biomarker is CRP.

5. The method of claim 2, wherein the biomarker is elevated WBC.

6. The method of claim 1, wherein said omega-3 fatty acid comprises docosahexaenoic acid (DHA).

7. The method of claim 6, wherein said omega-3 fatty acid comprises DHA and EPA in a ratio of greater than 10:1 (DHA:EPA).

8. The method of claim 6, wherein said DHA is essentially free of eicosapentaenoic acid (EPA).

9. The method of claim 1 wherein the non-alpha-tocopherol is selected from the group consisting of gamma-tocopherol, a gamma-tocopherol metabolite, beta-tocopherol, a beta-tocopherol metabolite, delta-tocopherol and delta-tocopherol metabolite.

10. The method of claim 9, wherein said non-alpha-tocopherol consists of a mixture of one or more a tocopherol selected from the group consisting of gamma-tocopherol, a gamma-tocopherol metabolite, beta-tocopherol, a beta-tocopherol metabolite, delta-tocopherol and a delta-tocopherol metabolite.

11. The method of claim 1, wherein said non-alpha-tocopherol is gamma-tocopherol.

12. The method of claim 1, wherein said non-alpha-tocopherol is gamma-carboxy ethyl hydroxy chroman (gamma-CEHC).

13. The method of claim 1, wherein said non-alpha-tocopherol is beta-tocopherol or a metabolite thereof.

14. The method of claim 1, wherein said non-alpha tocopherol is delta-tocopherol or a metabolite thereof.

15. The method of claim 1, wherein said formulation further comprises a flavonoid.

16. The method of claim 15, wherein said flavonoid is selected from the group consisting of quercetin, hesperetin, or a mixture of quercetin and hesperetin.

17. The method of claim 1, wherein said formulation further comprises a mineral component.

18. The method of claim 17, wherein said mineral component is magnesium.

19. The method of claim 1, wherein said inflammatory condition is muscle inflammation.

20. The method of claim 1, wherein said inflammatory condition is end-stage renal disease (ESRD).

21. The method of claim 1, wherein said inflammatory condition is diabetes.

22. The method of claim 1, wherein said inflammatory condition is cardiovascular disease.

23. The method of claim 1, wherein said inflammatory condition is metabolic syndrome.

24. A method for ameliorating a symptom of an inflammatory condition in an individual subject to an inflammatory condition comprising administering to the individual an effective amount of a formulation comprising a non-alpha-tocopherol and an omega-3 fatty acid.

25. The method of claim 24, wherein said symptom is elevation of a biomarker selected from the group consisting of C-reactive protein (CRP), interleukin-1-alpha (IL-1-alpha), interleukin-1-beta (IL-1-beta), interleukin-6 (IL-6), and white blood cell count (WBC).

26. The method of claim 25, wherein the biomarker is IL-6

27. The method of claim 25, wherein the biomarker is CRP.

28. The method of claim 25, wherein the biomarker is WBC.

29. The method of claim 25, wherein the symptom is edema.

30. The method of claim 25, wherein the non-alpha-tocopherol is selected from the group consisting of gamma-tocopherol, a gamma-tocopherol metabolite, beta-tocopherol, a beta-tocopherol metabolite, delta-tocopherol and delta-tocopherol metabolite.

31. The method of claim 24, wherein said omega-3 fatty acid comprises docosahexaenoic acid (DHA).

32. The method of claim 31, wherein said omega-3 fatty acid is essentially free of eicosapentaenoic acid (EPA).

33. The method of claim 24, wherein said omega-3 fatty acid comprises DHA and EPA in a ratio of greater than 10:1 (DHA:EPA).

34. The method of claim 24, wherein non-alpha-tocopherol is gamma-tocopherol.

35. The method of claim 24, wherein said non-alpha-tocopherol is gamma-carboxy ethyl hydroxy chroman (gamma-CEHC).

36. The method of claim 23, wherein said non-alpha-tocopherol is beta-tocopherol or a metabolite thereof.

37. The method of claim 24, wherein said non-alpha tocopherol is delta-tocopherol or a metabolite thereof.

38. The method of claim 24, wherein said formulation further comprises a flavonoid.

39. The method of claim 38, wherein said flavonoid is selected from the group consisting of quercetin, hesperetin, or a mixture of quercetin and hesperetin.

40. The method of claim 24, wherein said formulation further comprises a mineral component.

41. The method of claim 24, wherein said mineral component is magnesium

42. The method of claim 24, wherein said inflammatory condition is muscle inflammation.

43. The method of claim 24, wherein said inflammatory condition is end-stage renal disease (ESRD).

44. The method of claim 24, wherein said inflammatory condition is diabetes.

45. The method of claim 24, wherein said inflammatory condition is cardiovascular disease.

46. The method of claim 24, wherein said inflammatory condition is metabolic syndrome.
